SEQUENCE LISTING (64 sequences)

SEQ ID NO: 1 - protein, 453 aa, Homo sapiens
MRVQNEGTGKSSWWIINPDGGKSGKAPRRRAVSMDNSNKYTKSRGRAAKKKAALQTAPESADDSPSQLSKWPGSPTSRSSDELDAWTDFRSRTNSNASTVSGRLSPIMASTELDEVQDDDAPLSPMLYSSSASLSPSVSKPCTVELPRLTDMAGTMNLNDGLTENLMDDLLDNITLPPSQPSPTGGLMQRSSSFPYTTKGSGLGSPTSSFNSTVFGPSSLNSLRQSPMQTIQENKPATFSSMSHYGNQTLQDLLTSDSLSHSDVMMTQSDPLMSQASTAVSAQNSRRNVMLRNDPMMSFAAQPNQGSLVNQNLLHHQHQTQGALGGSRALSNSVSNMGLSESSSLGSAKHQQQSPVSQSMQTLSDSLSGSSLYSTSANLPVMGHEKFPSDLDLDMFNGSLECDMESIIRSELMDADGLDFNFDSLISTQNVVGLNVGNFTGAKQASSQSWVPG

SEQ ID NO: 2 - DNA, 1362 bp, Homo sapiens
atgcgggtccagaatgagggaactggcaagagctcttggtggatcatcaaccctgatggggggaagagcggaaaagccccccggcggcgggctgtctccatggacaatagcaacaagtataccaagagccgtggccgcgcagccaagaagaaggcagccctgcagacagcccccgaatcagctgacgacagtccctcccagctctccaagtggcctggcagccccacgtcacgcagcagtgatgagctggatgcgtggacggacttccgttcacgcaccaattctaacgccagcacagtcagtggccgcctgtcgcccatcatggcaagcacagagttggatgaagtccaggacgatgatgcgcctctctcgcccatgctctacagcagctcagccagcctgtcaccttcagtaagcaagccgtgcacggtggaactgccacggctgactgatatggcaggcaccatgaatctgaatgatgggctgactgaaaacctcatggacgacctgctggataacatcacgctcccgccatcccagccatcgcccactgggggactcatgcagcggagctctagcttcccgtataccaccaagggctcgggcctgggctccccaaccagctcctttaacagcacggtgttcggaccttcatctctgaactccctacgccagtctcccatgcagaccatccaagagaacaagccagctaccttctcttccatgtcacactatggtaaccagacactccaggacctgctcacttcggactcacttagccacagcgatgtcatgatgacacagtcggaccccttgatgtctcaggccagcaccgctgtgtctgcccagaattcccgccggaacgtgatgcttcgcaatgatccgatgatgtcctttgctgcccagcctaaccagggaagtttggtcaatcagaacttgctccaccaccagcaccaaacccagggcgctcttggtggcagccgtgccttgtcgaattctgtcagcaacatgggcttgagtgagtccagcagccttgggtcagccaaacaccagcagcagtctcctgtcagccagtctatgcaaaccctctcggactctctctcaggctcctccttgtactcaactagtgcaaacctgcccgtcatgggccatgagaagttccccagcgacttggacctggacatgttcaatgggagcttggaatgtgacatggagtccattatccgtagtgaactcatggatgctgatgggttggattttaactttgattccctcatctccacacagaatgttgttggtttgaacgtggggaacttcactggtgctaagcaggcctcatctcagagctgggtgccaggctga

SEQ ID NO: 3 - protein, 673 aa, Homo sapiens
MAEAPASPAPLSPLEVELDPEFEPQSRPRSCTWPLQRPELQASPAKPSGETAADSMIPEEEDDEDDEDGGGRAGSAMAIGGGGGSGTLGSGLLLEDSARVLAPGGQDPGSGPATAAGGLSGGTQALLQPQQPLPPPQPGAAGGSGQPRKCSSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRAVSMDNSNKYTKSRGRAAKKKAALQTAPESADDSPSQLSKWPGSPTSRSSDELDAWTDFRSRTNSNASTVSGRLSPIMASTELDEVQDDDAPLSPMLYSSSASLSPSVSKPCTVELPRLTDMAGTMNLNDGLTENLMDDLLDNITLPPSQPSPTGGLMQRSSSFPYTTKGSGLGSPTSSFNSTVFGPSSLNSLRQSPMQTIQENKPATFSSMSHYGNQTLQDLLTSDSLSHSDVMMTQSDPLMSQASTAVSAQNSRRNVMLRNDPMMSFAAQPNQGSLVNQNLLHHQHQTQGALGGSRALSNSVSNMGLSESSSLGSAKHQQQSPVSQSMQTLSDSLSGSSLYSTSANLPVMGHEKFPSDLDLDMFNGSLECDMESIIRSELMDADGLDFNFDSLISTQNVVGLNVGNFTGAKQASSQSWVPG

SEQ ID NO: 4 - DNA, 2022 bp, Homo sapiens
atggcagaggcaccggcttccccggccccgctctctccgctcgaagtggagctggacccggagttcgagccccagagccgtccgcgatcctgtacgtggcccctgcaaaggccggagctccaagcgagccctgccaagccctcgggggagacggccgccgactccatgatccccgaggaggaggacgatgaagacgacgaggacggcgggggacgggccggctcggccatggcgatcggcggcggcggcgggagcggcacgctgggctccgggctgctccttgaggactcggcccgggtgctggcacccggagggcaagaccccgggtctgggccagccaccgcggcgggcgggctgagcgggggtacacaggcgctgctgcagcctcagcaaccgctgccaccgccgcagccgggggcggctgggggctccgggcagccgaggaaatgttcgtcgcggcggaacgcctggggaaacctgtcctacgcggacctgatcacccgcgccatcgagagctccccggacaaacggctcactctgtcccagatctacgagtggatggtgcgttgcgtgccctacttcaaggataagggcgacagcaacagctctgccggctggaagaactccatccggcacaacctgtcactgcatagtcgattcatgcgggtccagaatgagggaactggcaagagctcttggtggatcatcaaccctgatggggggaagagcggaaaagccccccggcggcgggctgtctccatggacaatagcaacaagtataccaagagccgtggccgcgcagccaagaagaaggcagccctgcagacagcccccgaatcagctgacgacagtccctcccagctctccaagtggcctggcagccccacgtcacgcagcagtgatgagctggatgcgtggacggacttccgttcacgcaccaattctaacgccagcacagtcagtggccgcctgtcgcccatcatggcaagcacagagttggatgaagtccaggacgatgatgcgcctctctcgcccatgctctacagcagctcagccagcctgtcaccttcagtaagcaagccgtgcacggtggaactgccacggctgactgatatggcaggcaccatgaatctgaatgatgggctgactgaaaacctcatggacgacctgctggataacatcacgctcccgccatcccagccatcgcccactgggggactcatgcagcggagctctagcttcccgtataccaccaagggctcgggcctgggctccccaaccagctcctttaacagcacggtgttcggaccttcatctctgaactccctacgccagtctcccatgcagaccatccaagagaacaagccagctaccttctcttccatgtcacactatggtaaccagacactccaggacctgctcacttcggactcacttagccacagcgatgtcatgatgacacagtcggaccccttgatgtctcaggccagcaccgctgtgtctgcccagaattcccgccggaacgtgatgcttcgcaatgatccgatgatgtcctttgctgcccagcctaaccagggaagtttggtcaatcagaacttgctccaccaccagcaccaaacccagggcgctcttggtggcagccgtgccttgtcgaattctgtcagcaacatgggcttgagtgagtccagcagccttgggtcagccaaacaccagcagcagtctcctgtcagccagtctatgcaaaccctctcggactctctctcaggctcctccttgtactcaactagtgcaaacctgcccgtcatgggccatgagaagttccccagcgacttggacctggacatgttcaatgggagcttggaatgtgacatggagtccattatccgtagtgaactcatggatgctgatgggttggattttaactttgattccctcatctccacacagaatgttgttggtttgaacgtggggaacttcactggtgctaagcaggcctcatctcagagctgggtgccaggctga

SEQ ID NO: 5 - protein, 453 aa, Mus musculus
MRVQNEGTGKSSWWIINPDGGKSGKAPRRRAVSMDNSNKYTKSRGRAAKKKAALQAAPESADDSPSQLSKWPGSPTSRSSDELDAWTDFRSRTNSNASTVSGRLSPILASTELDDVQDDDGPLSPMLYSSSASLSPSVSKPCTVELPRLTDMAGTMNLNDGLAENLMDDLLDNIALPPSQPSPPGGLMQRGSSFPYTAKSSGLGSPTGSFNSTVFGPSSLNSLRQSPMQTIQENRPATFSSVSHYGNQTLQDLLASDSLSHSDVMMTQSDPLMSQASTAVSAQNARRNVMLRNDPMMSFAAQPTQGSLVNQNLLHHQHQTQGALGGSRALSNSVSNMGLSDSSSLGSAKHQQQSPASQSMQTLSDSLSGSSLYSASANLPVMGHDKFPSDLDLDMFNGSLECDMESIIRSELMDADGLDFNFDSLISTQNVVGLNVGNFTGAKQASSQSWVPG

SEQ ID NO: 6 - DNA, 1362 bp, Mus musculus
atgcgcgttcagaatgaaggcacgggcaagagctcttggtggatcatcaaccccgatgggggaaagagcgggaaggccccccggcggcgtgcggtctccatggacaacagcaacaagtacaccaagagccgaggccgggcagccaagaagaaggcggccctgcaggctgccccagagtcggcagacgacagtccttcccagctctccaagtggcctggcagccccacgtcccgcagcagcgacgagctggatgcgtggaccgacttccgctcgcgcaccaattccaacgccagcaccgtgagcggccgcctgtcgcccatcctggcaagcacggagctggatgacgtccaggatgatgatggacccctgtcccccatgctgtacagcagctctgccagcctgtcgccctccgtgagcaagccgtgtactgtggagcttccgcggctgacggacatggccggcaccatgaatctgaatgatgggctggccgagaacctcatggacgacctgctggataacatcgcgctcccgccatcgcagccatcgcctcctggcgggcttatgcagcggggctccagcttcccatataccgccaagagctccggcctgggctccccaaccggctccttcaacagtaccgtgtttggaccttcgtctctgaactccttgcgtcagtcacccatgcagactatccaggagaacagaccagccaccttctcttccgtgtcacactacggcaaccagacactccaagacctgcttgcttcagactcactcagccacagcgacgtcatgatgacccagtcggaccccttgatgtctcaggctagcaccgccgtgtccgcccagaatgcccgccggaacgtgatgcttcgcaacgatccaatgatgtcctttgctgcccagcctacccaggggagtttggtcaatcagaacttgctccaccaccagcaccaaacccagggcgctcttggtggcagccgtgccttgtcaaattctgtcagcaacatgggcttgagtgactccagcagccttggctcagccaaacaccagcagcagtctcccgccagccagtctatgcaaaccctctcggactctctctcaggctcctcactgtattcagctagtgcaaaccttcccgtcatgggccacgataagttccccagtgacttggacctggacatgttcaatgggagcttggaatgtgacatggagtccatcatccgtagtgaactcatggatgctgacgggttggattttaactttgactccctcatctccacacagaacgttgttggtttgaatgtggggaacttcactggtgctaagcaggcctcatctcaaagctgggtaccaggctga

SEQ ID NO: 7 - protein, 672 aa, Mus musculus
MAEAPASPVPLSPLEVELDPEFEPQSRPRSCTWPLQRPELQASPAKPSGETAADSMIPEEDDDEDDEDGGGRASSAMVIGGGVSSTLGSGLLLEDSAMLLAPGGQDLGSGPASAAGALSGGTPTQLQPQQPLPQPQPGAAGGSGQPRKCSSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRAVSMDNSNKYTKSRGRAAKKKAALQAAPESADDSPSQLSKWPGSPTSRSSDELDAWTDFRSRTNSNASTVSGRLSPILASTELDDVQDDDGPLSPMLYSSSASLSPSVSKPCTVELPRLTDMAGTMNLNDGLAENLMDDLLDNIALPPSQPSPPGGLMQRGSSFPYTAKSSGLGSPTGSFNSTVFGPSSLNSLRQSPMQTIQENRPATFSSVSHYGNQTLQDLLASDSLSHSDVMMTQSDPLMSQASTAVSAQNARRNVMLRNDPMMSFAAQPTQGSLVNQNLLHHQHQTQGALGGSRALSNSVSNMGLSDSSSLGSAKHQQQSPASQSMQTLSDSLSGSSLYSASANLPVMGHDKFPSDLDLDMFNGSLECDMESIIRSELMDADGLDFNFDSLISTQNVVGLNVGNFTGAKQASSQSWVPG

SEQ ID NO: 8 - DNA, 2019 bp, Mus musculus
atggcagaggcaccagcctccccggtcccgctctctccgctcgaagtggagctggacccagagttcgagccacagagtcggccacgctcctgtacgtggcccctgcagaggccggagctgcaggcgagcccggccaagccctcgggggagacggccgcagactccatgatccccgaggaggacgacgatgaagacgacgaggacggcggcggccgagccagctcggccatggtgatcggtggcggcgtgagcagcacgctgggttccgggctgctcctcgaggattcggccatgctgctggctccaggagggcaggacctcgggtcggggccagcgtccgccgcaggcgctctgagtgggggcacgccgacgcagctgcagcctcagcagccactgccacagccgcagccgggggcggctgggggctctgggcaaccaaggaaatgctcctcgcggcggaatgcctgggggaacctgtcctatgccgacctgatcacccgcgccatcgagagctccccggacaaacggctcactttgtcccagatctacgagtggatggtgcgctgtgtgccctacttcaaggataagggcgacagcaacagctctgcgggctggaagaactccatccggcacaacctgtccctgcacagccgcttcatgcgcgttcagaatgaaggcacgggcaagagctcttggtggatcatcaaccccgatgggggaaagagcgggaaggccccccggcggcgtgcggtctccatggacaacagcaacaagtacaccaagagccgaggccgggcagccaagaagaaggcggccctgcaggctgccccagagtcggcagacgacagtccttcccagctctccaagtggcctggcagccccacgtcccgcagcagcgacgagctggatgcgtggaccgacttccgctcgcgcaccaattccaacgccagcaccgtgagcggccgcctgtcgcccatcctggcaagcacggagctggatgacgtccaggatgatgatggacccctgtcccccatgctgtacagcagctctgccagcctgtcgccctccgtgagcaagccgtgtactgtggagcttccgcggctgacggacatggccggcaccatgaatctgaatgatgggctggccgagaacctcatggacgacctgctggataacatcgcgctcccgccatcgcagccatcgcctcctggcgggcttatgcagcggggctccagcttcccatataccgccaagagctccggcctgggctccccaaccggctccttcaacagtaccgtgtttggaccttcgtctctgaactccttgcgtcagtcacccatgcagactatccaggagaacagaccagccaccttctcttccgtgtcacactacggcaaccagacactccaagacctgcttgcttcagactcactcagccacagcgacgtcatgatgacccagtcggaccccttgatgtctcaggctagcaccgccgtgtccgcccagaatgcccgccggaacgtgatgcttcgcaacgatccaatgatgtcctttgctgcccagcctacccaggggagtttggtcaatcagaacttgctccaccaccagcaccaaacccagggcgctcttggtggcagccgtgccttgtcaaattctgtcagcaacatgggcttgagtgactccagcagccttggctcagccaaacaccagcagcagtctcccgccagccagtctatgcaaaccctctcggactctctctcaggctcctcactgtattcagctagtgcaaaccttcccgtcatgggccacgataagttccccagtgacttggacctggacatgttcaatgggagcttggaatgtgacatggagtccatcatccgtagtgaactcatggatgctgacgggttggattttaactttgactccctcatctccacacagaacgttgttggtttgaatgtggggaacttcactggtgctaagcaggcctcatctcaaagctgggtaccaggctga

SEQ ID NO: 9 - DNA, 32 bp, Artificial Sequence
  Primer
attctagagccaccatggcagaggcaccagcc

SEQ ID NO: 10 - DNA, 54 bp, Artificial Sequence
  Primer
atggatcctcacttgtcgtcatcgtctttgtagtcgcctggtacccagctttga

SEQ ID NO: 11 - DNA, 32 bp, Artificial Sequence; identical to SEQ ID NO: 9
  Primer

SEQ ID NO: 12 - DNA, 53 bp, Artificial Sequence
  Primer
atggatcctcacttgtcgtcatcgtctttgtagtccttccagcccgcagagct

SEQ ID NO: 13 - DNA, 34 bp, Artificial Sequence
  Primer
attctagagccaccatgcgcgttcagaatgaagg

SEQ ID NO: 14 - DNA, 54 bp, Artificial Sequence; identical to SEQ ID NO: 10
  Primer

SEQ ID NO: 15 - DNA, 18 bp, Artificial Sequence
  Primer
acggctacttgcggtttc

SEQ ID NO: 16 - DNA, 17 bp, Artificial Sequence
  Primer
tccttgggaggctggtc

SEQ ID NO: 17 - DNA, 22 bp, Artificial Sequence
  Primer
tttgccgctgtggactatctgc

SEQ ID NO: 18 - DNA, 24 bp, Artificial Sequence
  Primer
agacgtggtttaggaatgcagctc

SEQ ID NO: 19 - DNA, 19 bp, Artificial Sequence
  Primer
aagatattggtggctttgg

SEQ ID NO: 20 - DNA, 16 bp, Artificial Sequence
  Primer
atcgctgcgtccctct

SEQ ID NO: 21 - DNA, 24 bp, Artificial Sequence
  Primer
ccgggggacttaatgagaccactt

SEQ ID NO: 22 - DNA, 23 bp, Artificial Sequence
  Primer
acgccccaaatcccacccataca

SEQ ID NO: 23 - DNA, 24 bp, Artificial Sequence
  Primer
acatgatccagttcaccaggcaga

SEQ ID NO: 24 - DNA, 24 bp, Artificial Sequence
  Primer
aggttcttggtgacctcccaactt

SEQ ID NO: 25 - DNA, 20 bp, Artificial Sequence
  Primer
ctgtcctatgccgacctgat

SEQ ID NO: 26 - DNA, 20 bp, Artificial Sequence
  Primer
ctgtcgcccttatccttgaa

SEQ ID NO: 27 - DNA, 20 bp, Artificial Sequence
  Primer
atgggagcttggaatgtgac

SEQ ID NO: 28 - DNA, 20 bp, Artificial Sequence
  Primer
ttaaaatccaacccgtcagc

SEQ ID NO: 29 - DNA, 20 bp, Artificial Sequence
  Primer
aggaggaggaatgtggaagg

SEQ ID NO: 30 - DNA, 19 bp, Artificial Sequence
  Primer
ccgtgccttcattctgaac

SEQ ID NO: 31 - DNA, 20 bp, Artificial Sequence
  Primer
ttacactgcctttgccatcc

SEQ ID NO: 32 - DNA, 20 bp, Artificial Sequence
  Primer
agaaacactgtctgctggtg

SEQ ID NO: 33 - DNA, 20 bp, Artificial Sequence
  Primer
ggcagacaccacatacaacc

SEQ ID NO: 34 - DNA, 19 bp, Artificial Sequence
  Primer
cctcaggctagatggcaag

SEQ ID NO: 35 - DNA, 20 bp, Artificial Sequence
  Primer
ctccactttcagagccttcg

SEQ ID NO: 36 - DNA, 20 bp, Artificial Sequence
  Primer
tgctgagatggactgtgagg

SEQ ID NO: 37 - DNA, 20 bp, Artificial Sequence
  Primer
gtctcggcttgtgcttgtct

SEQ ID NO: 38 - DNA, 20 bp, Artificial Sequence
  Primer
ccaggtccatgaggaagtgt

SEQ ID NO: 39 - DNA, 22 bp, Artificial Sequence
  Primer
aaatgtcatgggtactggagtt

SEQ ID NO: 40 - DNA, 22 bp, Artificial Sequence
  Primer
atggcaattatcaagtttgtgg

SEQ ID NO: 41 - DNA, 22 bp, Artificial Sequence
  Primer
cagatattattcgcaactacaa

SEQ ID NO: 42 - DNA, 20 bp, Artificial Sequence
  Primer
tggggtacagatacttcagg

SEQ ID NO: 43 - DNA, 20 bp, Artificial Sequence
  Primer
atcaagaaggtggtgaagca

SEQ ID NO: 44 - DNA, 21 bp, Artificial Sequence
  Primer
agacaacctggtcctcagtgt

SEQ ID NO: 45 - DNA, 20 bp, Artificial Sequence
  Primer
ttcaaggataagggcgacag

SEQ ID NO: 46 - DNA, 20 bp, Artificial Sequence
  Primer
cctcggctcttggtgtactt

SEQ ID NO: 47 - DNA, 20 bp, Artificial Sequence
  Primer
cgttgttggtttgaatgtgg

SEQ ID NO: 48 - DNA, 20 bp, Artificial Sequence
  Primer
cgtgggagtctcaaaggtgt

SEQ ID NO: 49 - DNA, 19 bp, Artificial Sequence
  Primer
atgcgcgttcagaatgaag

SEQ ID NO: 50 - DNA, 20 bp, Artificial Sequence
  Primer
ggagagctgggaaggactgt

SEQ ID NO: 51 - DNA, 20 bp, Artificial Sequence
  Primer
ccatggacaacagcaacaag

SEQ ID NO: 52 - DNA, 20 bp, Artificial Sequence
  Primer
cagcccatcattcagattca

SEQ ID NO: 53 - DNA, 20 bp, Artificial Sequence
  Primer
gatgatgatggacccctgtc

SEQ ID NO: 54 - DNA, 20 bp, Artificial Sequence
  Primer
gaagcaagcaggtcttggag

SEQ ID NO: 55 - DNA, 20 bp, Artificial Sequence
  Primer
ggggagtttggtcaatcaga

SEQ ID NO: 56 - DNA, 20 bp, Artificial Sequence
  Primer
ttaaaatccaacccgtcagc

SEQ ID NO: 57 - DNA, 20 bp, Artificial Sequence
  Primer
ctgtcctatgccgacctgat

SEQ ID NO: 58 - DNA, 20 bp, Artificial Sequence
  Primer
ctgtcgcccttatccttgaa

SEQ ID NO: 59 - DNA, 20 bp, Artificial Sequence
  Primer
atgggagcttggaatgtgac

SEQ ID NO: 60 - DNA, 20 bp, Artificial Sequence
  Primer
ttaaaatccaacccgtcagc

SEQ ID NO: 61 - DNA, 20 bp, Artificial Sequence
  Primer
aggaggaggaatgtggaagg

SEQ ID NO: 62 - DNA, 19 bp, Artificial Sequence
  Primer
ccgtgccttcattctgaac

SEQ ID NO: 63 - DNA, 27 bp, Artificial Sequence
  Primer
attctagactaggttgaggctccctgt

SEQ ID NO: 64 - DNA, 30 bp, Artificial Sequence
  primer
attccggatccgcctggtacccagctttga

(19) United States
(12) Patent Application Publication
(10) Pub. No.: US 2022/0305080 A1
(43) Pub. Date: Sep. 29, 2022

(54) COMPOSITIONS AND METHODS UTILIZING A NOVEL HUMAN FOXO3 ISOFORM

(71) Applicant: New York Society for the Relief of the Ruptured and Crippled, Maintaining the Hospital for Special, New York, NY (US)

(72) Inventor: Baohong Zhao, New York, NY (US)

(21) Appl. No.: 17/635,594

(22) PCT Filed: Aug. 14, 2020
(86) PCT No.: PCT/US20/46292

§ 371 Date: Feb. 15, 2022

Related U.S. Application Data
(60) Provisional application No. 62/888,162, filed on Aug. 16, 2019.

(51) Int. Cl. A61K 38/17; G01N 33/68; A61P 19/00

(52) U.S. Cl. CPC A61K 38/1709; G01N 33/6893; A61P 19/00; G01N 2333/4703; G01N 2800/52; G01N 2800/50; G01N 2800/108

(57) ABSTRACT

Provided herein is a method of suppressing osteoclast differentiation or function and/or bone resorption or destruction in a subject in need thereof and compositions therefore. In one embodiment, the method includes increasing the amount, expression, or activity of Foxo3 isoform 2 in the subject.

### STATEMENT REGARDING FEDERALLY SPONSORED RESEARCH OR DEVELOPMENT

This invention was made with government support under AR062047, AR068970, and AR071463 awarded by the National Institutes of Health. The government has certain rights in the invention.

### BACKGROUND

Osteoclasts, derived from monocyte/macrophage precursors, are the exclusive cell type responsible for bone resorption in both bone homeostasis and pathological bone destruction. Bone loss is a major cause of morbidity and disability in many skeletal diseases, such as rheumatoid arthritis (RA), psoriatic arthritis, periodontitis, and periprosthetic loosening (Novack, D. V., and S. L. Teitelbaum. 2008. The osteoclast: friend or foe? Annu. Rev. Pathol. 3: 457-484; Sato, K., and H. Takayanagi. 2006. Osteoclasts, rheumatoid arthritis, and osteoimmunology. Curr. Opin. Rheumatol. 18: 419-426; Schett, G., and E. Gravallese. 2012. Bone erosion in rheumatoid arthritis: mechanisms, diagnosis and treatment. Nat. Rev. Rheumatol. 8: 656-664, all incorporated herein by reference). Osteoclastogenesis is induced by the major osteoclastogenic cytokine receptor activator of NF-κB ligand (RANKL). Binding of RANKL to RANK receptors activates a broad range of signaling cascades, including canonical and noncanonical NF-κB pathways, MAPK pathways, and calcium signaling, which lead to the activation of an osteoclastic transcriptional network. The positive regulators in this transcriptional network, such as the transcription factors NFATc1, c-Fos, and Blimp1, drive osteoclast differentiation (Asagiri, M., and H. Takayanagi. 2007. The molecular understanding of osteoclast differentiation. Bone 40: 251-264.). In contrast, the process of osteoclast differentiation is delicately controlled by a “braking system,” in which negative regulators, such as IFN regulatory factor (Irf) 8, recombination signal binding protein for Ig k J region (RBP-J), and differentially expressed in FDCP 6 homolog (Def6), restrain osteoclastogenesis to prevent excessive bone resorption (Binder, N., C. Miller, M. Yoshida, K. Inoue, S. Nakano, X. Hu, L. B. Ivashkiv, G. Schett, A. Pernis, S. R. Goldring, et al. 2017. Def6 restrains osteoclastogenesis and inflammatory bone resorption. J. Immunol. 198: 3436-3447; Li, S., C. H. Miller, E. Giannopoulou, X. Hu, L. B. Ivashkiv, and B. Zhao. 2014. RBP-J imposes a requirement for ITAM-mediated costimulation of osteoclastogenesis. J. Clin. Invest. 124: 5057-5073; Zhao, B., S. N. Grimes, S. Li, X. Hu, and L. B. Ivashkiv. 2012. TNF-induced osteoclastogenesis and inflammatory bone resorption are inhibited by transcription factor RBP-J. J. Exp. Med. 209: 319-334; Zhao, B., and L. B. Ivashkiv. 2011. Negative regulation of osteoclastogenesis and bone resorption by cytokines and transcriptional repressors. Arthritis Res. Ther. 13: 234; Zhao, B., M. Takami, A. Yamada, X. Wang, T. Koga, X. Hu, T. Tamura, K. Ozato, Y. Choi, L. B. Ivashkiv, et al. 2009. Interferon regulatory factor-8 regulates bone metabolism by suppressing osteoclastogenesis. Nat. Med. 15: 1066-1071, all incorporated herein by reference). Thus, the extent of osteoclastogenesis is delicately modulated and determined by the balance between these osteoclastogenic and antiosteoclastogenic mechanisms.

Forkhead box class 0 (Foxo) proteins are a family of evolutionarily conserved transcription factors, which include Foxo1, 3, 4, and 6 in mammals. Foxo proteins consist of four conserved regions: a forkhead DNA-binding domain at the N terminus followed by a nuclear localization signal, a nuclear export signal, and a transactivation domain at the C terminus (Hedrick, S. M., R. Hess Michelini, A. L. Doedens, A. W. Goldrath, and E. L. Stone. 2012. FOXO transcription factors throughout T cell biology. Nat. Rev. Immunol. 12: 649-661; Tia, N., A. K. Singh, P. Pandey, C. S. Azad, P. Chaudhary, and I. S. Gambhir. 2018. Role of Forkhead Box 0 (FOXO) transcription factor in aging and diseases. Gene 648: 97-105; Wang, X., S. Hu, and L. Liu. 2017. Phosphorylation and acetylation modifications of FOXO3a: independently or synergistically? Oncol. Lett. 13: 2867-2872, all incorporated herein by reference). Foxo proteins play important roles in diverse biological processes, such as metabolism, oxidative stress, cell cycle regulation, apoptosis, immunity, and inflammation. Foxo proteins are well known for their cell type- and context-specific effects on cellular processes because of their variable posttranslational modifications, subcellular localization, and binding cofactors in different scenarios (Salih, D. A., and A. Brunet. 2008. FoxO transcription factors in the maintenance of cellular homeostasis during aging. Curr. Opin. Cell Biol. 20: 126-136; van der Vos, K. E., and P. J. Coffer. 2008. FOXO-binding partners: it takes two to tango. Oncogene 27: 2289-2299. Morris, B. J., D. C. Willcox, T. A. Donlon, and B. J. Willcox. 2015. FOXO3: a major gene for human longevity—A mini-review. Gerontology 61: 515-525, all incorporated herein by reference). Foxo1, 3, and 4 were reported to regulate RANKL-induced osteoclast differentiation (Bartell, S. M., H. N. Kim, E. Ambrogini, L. Han, S. Iyer, S. Serra Ucer, P. Rabinovitch, R. L. Jilka, R. S. Weinstein, H. Zhao, et al. 2014. FoxO proteins restrain osteoclastogenesis and bone resorption by attenuating H2O2 accumulation. Nat. Commun. 5: 3773; Wang, Y., G. Dong, H. H. Jeon, M. Elazizi, L. B. La, A. Hameedaldeen, E. Xiao, C. Tian, S. Alsadun, Y. Choi, and D. T. Graves. 2015. FOXO1 mediates RANKL-induced osteoclast formation and activity. J. Immunol. 194: 2878-2887, both incorporated herein by reference).

However, Foxo proteins seem to exhibit different functions in osteoclastogenesis. For example, some studies show that Foxo1, 3, and 4 proteins as a group are inhibitors of osteoclastogenesis (Bartell 2014), whereas others found that Foxo1 is a positive regulator (Wang 2015). These results indicate that Foxo family plays an important but complex role in osteoclastogenesis. In disease settings, FOXO3 activity is correlated with outcomes in infectious and inflammatory diseases, such as RA. Increased expression of FOXO3 in monocytes due to a single-nucleotide polymorphism (FOXO3 [rs12212067: T.G]) is associated with reduced severity of RA (Gregersen, P. K., and N. Manjarrez-Orduño. 2013. FOXO in the hole: leveraging GWAS for outcome and function. Cell 155: 11-12; Lee, J. C., M. Espe′li, C. A. Anderson, M. A. Linterman, J. M. Pocock, N. J. Williams, R. Roberts, S. Viatte, B. Fu, N. Peshu, et al; UK IBD Genetics Consortium. 2013. Human SNP links differential outcomes in inflammatory and infectious disease to a FOXO3-regulated pathway. Cell 155: 57-69., both incorporated herein by reference). Recently, it was uncovered that Foxo3 is a target of miR-182 and plays an inhibitory role in inflammatory cytokine TNF-a-induced osteoclastogenesis and bone resorption (Miller, C. H., S. M. Smith, M. Elguindy, T. Zhang, J. Z. Xiang, X. Hu, L. B. Ivashkiv, and B. Zhao. 2016. RBP-J-regulated miR-182 promotes TNF-a-induced osteoclastogenesis. J. Immunol. 196: 4977-4986, incorporated herein by reference). Thus, FOXO3 is closely involved in osteoclastogenesis and bone erosion in human RA.

What is needed are biomarkers and therapeutic targets for skeleton diseases.

### SUMMARY OF THE INVENTION

Provided herein, in one aspect is a method of suppressing osteoclast differentiation or function and/or bone resorption or destruction in a subject in need thereof. The method includes increasing the amount, expression, or activity of Foxo3 isoform 2 in the subject. In another aspect, a method of treating a skeletal disease in a subject in need thereof is provided. The method includes increasing the amount, expression, or activity of Foxo3 isoform 2 in the subject. In one embodiment of the methods described herein, Foxo3 isoform 2 has the sequence of SEQ ID NO: 1 or a sequence sharing at least 90% identity therewith. In one embodiment, the method includes administering an agonist of Foxo3 isoform 2, or a functional fragment thereof. In another embodiment, the method includes administering a nucleic acid which comprises a sequence encoding Foxo3 isoform 2 having the sequence of SEQ ID NO: 1 or a sequence sharing at least 90% identity therewith, or a functional fragment of Foxo3 isoform 2, having a N-terminal truncation and sharing at least 90% identity with SEQ ID NO: 1. In yet another embodiment, the method includes administering a polypeptide having the sequence of SEQ ID NO: 1 or a sequence sharing at least 90% identity therewith, or a functional fragment of Foxo3 isoform 2, having a N-terminal truncation and sharing at least 90% identity with SEQ ID NO: 1.

In another aspect, a pharmaceutical composition is provided. In one embodiment, the composition comprises a pharmaceutically acceptable carrier, diluent, or excipient and a viral vector comprising a nucleic acid which comprises a sequence encoding Foxo3 isoform 2 or a sequence sharing at least 90% identity therewith, or a functional fragment of Foxo3 isoform 2, having a N-terminal truncation and sharing at least 90% identity with SEQ ID NO: 1. In another embodiment, the composition comprises a pharmaceutically acceptable carrier, diluent, or excipient and a polypeptide having the sequence of SEQ ID NO: 1 or a sequence sharing at least 90% identity therewith, or a functional fragment of Foxo3 isoform 2, having a N-terminal truncation and sharing at least 90% identity with SEQ ID NO: 1.

In another aspect, a method of assessing the efficacy of a treatment is provided. The method includes measuring the level of Foxo3 isoform 2 in the blood of a subject receiving treatment, wherein an increase in the level of Foxo3 isoform 2 indicates effectiveness of the treatment for treating a skeletal disease.

In another aspect, a method of diagnosing an increased risk of developing a skeletal disease in a subject. The method includes measuring the level of Foxo3 isoform 2 in the blood of a subject receiving treatment, wherein a decrease in the level of Foxo3 isoform 2 as compared to a control level indicates a greater risk of developing a skeletal disease. In one embodiment, the method includes treating the subject for the skeletal disease.

In another aspect, a method of diagnosing a skeletal disease in a subject is provided. The method includes measuring the level of Foxo3 isoform 2 in the blood of a subject receiving treatment, wherein a decrease in the level of Foxo3 isoform 2 as compared to a control level indicates the presence of a skeletal disease.

Other aspects and advantages of the invention will be readily apparent from the following detailed description of the invention.

### DESCRIPTION OF THE FIGURES

FIGS. 1A-IC demonstrate that RANKL-induced osteoclast differentiation is enhanced by Foxo3 deficiency. Bone marrow macrophages (BMMs) derived from WT control and Foxo3 KO mice were stimulated with RANKL for 4 d. TRAP staining was performed (FIG. 1A), and the number of TRAP-positive multinucleated cells per well is shown in (FIG. 1B). TRAP positive cells appear dark in the photographs. Scale bar, 100 mm. Data are representative of three independent experiments. FIG. 1C is a heat map of RANKL-induced osteoclastic gene expression enhanced by Foxo3 deficiency. Row z-scores of CPMs of osteoclast genes are shown in the heat map. **p<0.01.

FIGS. 2A-2G demonstrate that Foxo3^{f/f};LysMcre (Foxo3^{isoform2}) mice express a truncated Foxo3 protein that is an ortholog of human FOXO3 isoform2. FIG. 2A shows the molecular structure of mouse Foxo3 and Loxp sites. FIG. 2B shows PCR primer locations in Foxo3. FIG. 2C is a gel showing Foxo3 gene expression detected in WT and Foxo3^{f/f};LysMcre BMMs by PCR using the indicated primer sets whose locations are shown in FIG. 2B. n=5 per group. FIG. 2D shows Foxo3 gene expression detected in WT and Foxo3^{f/f};LysMcre BMMs by quantitative PCR using the indicated primer sets whose locations are shown in FIG. 2A. FIG. 2E and FIG. 2F show a map of transcripts from primer set Exon 1F and Exon 3R for WT BMMs (FIG. 2E) and Foxo3^{f/f};LysMcre BMMs (FIG. 2F). FIG. 2G shows Foxo3 protein expression detected in WT and Foxo3^{f/f};LysMcre BMMs by Western blot using Abs recognizing C terminus or exon 2 of Foxo3, respectively. p38 was used as a loading control. All the primer sequences are shown in Table I.

FIGS. 3A-3E show mouse Foxo3 isoform2 suppresses osteoclastogenesis and leads to the osteopetrotic phenotype in mice. BMMs derived from WT control and Foxo3^{isoform2} mice which were stimulated with RANKL for 4 d. TRAP staining was performed (FIG. 3A), and the number of TRAP-positive multinucleated cells (MNCs) per well is shown in FIG. 3B). Scale bar, 100 mm. Data are representative of and statistical analysis was performed on three independent experiments. mCT images (FIG. 3C) and bone morphometric analysis (FIG. 3D) are of trabecular bone of the distal femurs isolated from the WT and Foxo3^{isoform2} mice. n=8 per group. FIG. 3E BMMs transfected with either control or Foxo3 siRNA (80 nM) were stimulated with RANKL for 5 d. The number of TRAP-positive MNCs (≥3 nuclei per cell) per well was calculated. *p<0.05, **p<0.01. BV/TV, bone volume per tissue volume; Tb.N, trabecular number; Tb.Sp, trabecular separation; Tb.Th, trabecular thickness.

FIGS. 4A-4D demonstrate that overexpression of Foxo3 isoform2 inhibits osteoclastogenesis. Immunoblot analysis of the expression of full-length Foxo3, Foxo3 isoform2, and exon 2 in whole cell lysates of HEK293 cells (FIG. 4A) or RAW264.7 cells (FIG. 4B) transfected with corresponding pcDNA3.1+ plasmids containing specific Foxo3 fragments as indicated in the Materials and Methods. Anti-Flag Ab was used in (A). In (B), Foxo3 C-terminal Ab was used to detect full-length Foxo3 and Foxo3 isoform2. Foxo3 N-terminal exon 2 Ab was used to detect Foxo3 exon 2. FIG. 4C shows RAW264.7 cells transfected with the indicated plasmids which were stimulated with RANKL for 6 d. TRAP staining was performed (data not shown), and the number of TRAP-positive multinucleated cells per well is shown. Scale bar, 100 mm. Data are representative of and statistical analysis was performed on three independent experiments. FIG. 4D shows results of Quantitative PCR analysis of the relative expression of CtsK and Acp5 induced by RANKL for 6 d in the RAW264.7 cells transfected with the indicated plasmids. The induction folds of gene expression by RANKL relative to each basal condition was calculated and is shown in the figure. Data are representative of three independent experiments. *p<0.05,**p<0.01.

FIGS. 5A and 5B show that mouse Foxo3 isoform2 suppresses osteoclastic gene expression but enhances type I IFN-responsive gene expression. BMMs derived from WT control and Foxo3^{isoform2} mice were stimulated with RANKL for 3 d. The expression of osteoclastic marker genes (FIG. 5A) and type I IFN response genes (FIG. 5B) was examined by quantitative PCR. Data are representative of three independent experiments. **p<0.01.

FIGS. 6A-6C show the molecular structure of human FOXO3 isoform2. FIG. 6A shows human FOXO3 isoform2 from RefSeq gene database shown in UCSC genome browser. FIG. 6B shows a comparison of the molecular structures between full-length FOXO3 and FOXO3 isoform2. FIG. 6C shows a comparison of the coding sequences (upper lanes) and amino acid sequences (lower lanes) between full-length FOXO3 and FOXO3 isoform2. SEQ ID NO: 1-hFoxo3 isoform 2 amino acid sequence; SEQ ID NO: 2-hFoxo3 isoform 2 coding sequence; SEQ ID NO: 3-full-length hFoxo3 isoform 1 amino acid sequence; SEQ ID NO: 4-full-length hFoxo3 isoform 1 nucleic acid sequence. Lighter text: FH domain.

FIG. 7 shows a comparison of the coding sequences (upper lanes) and amino acid sequences (lower lanes) between mouse (left) and human (right) full-length FOXO3. SEQ ID NO: 3-full-length hFoxo3 isoform 1 amino acid sequence; SEQ ID NO: 4-full-length hFoxo3 isoform 1 nucleic acid sequence. SEQ ID NO: 7-full-length mFoxo3 isoform 1 amino acid sequence; SEQ ID NO: 8-full-length mFoxo3 isoform 1 coding sequence.

FIG. 8 shows a comparison of the coding sequences (upper lanes) and amino acid sequences (lower lanes) between mouse (right) and human (left) FOXO3 isoform2. SEQ ID NO: 1-hFoxo3 isoform 2 amino acid sequence; SEQ ID NO: 2-hFoxo3 isoform 2 coding sequence; SEQ ID NO: 5-mFoxo3 isoform 2 amino acid sequence; SEQ ID NO: 6-mFoxo3 isoform 2 coding sequence.

### DETAILED DESCRIPTION OF THE INVENTION

Foxo3 acts as an important central regulator that integrates signaling pathways and coordinates cellular responses to environmental changes. Recent studies show the involvement of Foxo3 in osteoclastogenesis and rheumatoid arthritis, which prompted further investigation of the FOXO3 locus. Several databases document a putative FOXO3 isoform2, an N-terminal truncated mutation of the full-length FOXO3. However, the biological function of FOXO3 isoform2 was previously unknown. As disclosed herein, a conditional allele of Foxo3 in mice was established that deletes the full-length Foxo3 except isoform2, a close ortholog of the human FOXO3 isoform2. Expression of Foxo3 isoform2 specifically in macrophage/osteoclast lineage suppresses osteoclastogenesis and leads to the osteopetrotic phenotype in mice. As described herein, mechanistically, Foxo3 isoform2 enhances the expression of type I IFN response genes to RANKL stimulation and thus inhibits osteoclastogenesis via endogenous IFN-β-mediated feedback inhibition. These findings identify the first known biological function of Foxo3 isoform2 that acts as a novel osteoclastic inhibitor in bone remodeling.

It is to be noted that the term “a” or “an” refers to one or more. As such, the terms “a” (or “an”), “one or more,” and “at least one” are used interchangeably herein.

While various embodiments in the specification are presented using “comprising” language, under other circumstances, a related embodiment is also intended to be interpreted and described using “consisting of” or “consisting essentially of” language. The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

“Upregulate” and “upregulation”, as used herein, refer to an elevation in the level of expression of a product of one or more genes in a cell or the cells of a tissue or organ.

As used herein, the term “agonist” refers to a compound that in combination with a receptor can produce a cellular response. An agonist may be a ligand that directly binds to the receptor. Alternatively an agonist may combine with a receptor indirectly by for example (a) forming a complex with another molecule that directly binds to the receptor, or (b) otherwise resulting in the modification of another compound so that the other compound directly binds to the receptor. The term “Foxo3 isoform 2 agonist” in particular includes any entity which agonizes Foxo3 isoform 2. This includes Foxo3 isoform 2 agonistic antibodies and fragments thereof, as well as small molecule agonists. The term also includes agonists of Foxo3 isoform 1.

A “subject” is a mammal, e.g., a human, mouse, rat, guinea pig, dog, cat, horse, cow, pig, or non-human primate, such as a monkey, chimpanzee, baboon or gorilla. The term “patient” may be used interchangeably with the term subject. In one embodiment, the subject is a human. The subject may be of any age, as determined by the health care provider. In certain embodiments described herein, the patient is a subject who has or is at risk of developing a skeletal disease. The subject may have been treated for a skeletal disease previously, or is currently being treated for the skeletal disease.

As used herein, the term “skeletal disease” or “skeletal disorder” refers to any condition associated with the bone or joints, including those associated with bone loss, bone fragility, or softening, or aberrant skeletal growth. Skeletal diseases include, without limitation, osteoporosis and osteopenia, rheumatoid arthritis, osteoarthritis, psoriatic arthritis, periodontitis, periprosthetic loosening, osteomalacia, hyperparathyroidism, Paget disease of bone, spondyloarthritis, and lupus.

“Sample” as used herein means any biological fluid or tissue that contains cells or tissue, including blood cells, fibroblasts, and skeletal muscle. In one embodiment, the sample is whole blood. In another embodiment, the sample is peripheral blood mononuclear cells (PBMC). Other useful biological samples include, without limitation, peripheral blood mononuclear cells, plasma, saliva, urine, synovial fluid, bone marrow, cerebrospinal fluid, vaginal mucus, cervical mucus, nasal secretions, sputum, semen, amniotic fluid, bronchoscopy sample, bronchoalveolar lavage fluid, and other cellular exudates from a patient having cancer. Such samples may further be diluted with saline, buffer or a physiologically acceptable diluent. Alternatively, such samples are concentrated by conventional means.

By “fragment” is intended a molecule consisting of only a part of the intact full-length polypeptide sequence and structure. The fragment can include a C terminal deletion, an N terminal deletion, and/or an internal deletion of the native polypeptide. In one embodiment, the fragment includes an N-terminal deletion of up to 5, 10, 15, 20, 25, 30, 35, 40 or 45 amino acids. A fragment will generally include at least about 5-10 contiguous amino acid residues of the full length molecule, preferably at least about 15-25 contiguous amino acid residues of the full length molecule, and most preferably at least about 20 50 or more contiguous amino acid residues of the full length molecule, or any integer between 5 amino acids and the full length sequence, provided that the fragment in question retains the ability to elicit the desired biological response, although not necessarily at the same level.

The terms “percent (%) identity”, “sequence identity”, “percent sequence identity”, or “percent identical” in the context of nucleic acid sequences refers to the bases in the two sequences which are the same when aligned for correspondence. The length of sequence identity comparison may be over the full-length of the full-length of a gene coding sequence, or a fragment of at least about 100 to 150 nucleotides, or as desired. However, identity among smaller fragments, e.g. of at least about nine nucleotides, usually at least about 20 to 24 nucleotides, at least about 28 to 32 nucleotides, at least about 36 or more nucleotides, may also be desired. Multiple sequence alignment programs are also available for nucleic acid sequences. Examples of such programs include, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 70 amino acids to about 100 amino acids, or a peptide fragment thereof or the corresponding nucleic acid sequence coding sequencers. A suitable amino acid fragment may be at least about 8 amino acids in length, and may be up to about 450 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

The term “derived from” is used to identify the original source of a molecule (e.g., murine or human) but is not meant to limit the method by which the molecule is made which can be, for example, by chemical synthesis or recombinant means.

As used herein, the term “a therapeutically effective amount” refers an amount sufficient to achieve the intended purpose. For example, an effective amount of an Foxo3 isoform 2 agonist is sufficient to decrease osteoclastogenesis or osteoclast function, bone resorption or destruction in a subject. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined by a skilled artisan according to established methods in the art.

As used herein, “disease”, “disorder” and “condition” are used interchangeably, to indicate an abnormal state in a subject.

Provided herein, in one aspect, are methods of suppressing osteoclast differentiation or function and/or bone resorption or destruction in a subject. As described herein, expression of Foxo3 isoform 2 in macrophage/osteoclast lineage suppresses osteoclastogenesis. Thus, provided herein, are methods of treating skeletal diseases associated with osteoclastic bone remodeling.

Over 90% of human genes are alternatively spliced to produce mRNA and protein isoforms, which may have shared, related, distinct, or even antagonistic functions. Alternative splicing is an essential biological process driving evolution and development. The isoforms resulting from alternative splicing contribute to transcriptomic and proteomic diversity and complexity in physiological conditions (Vacik, T., and I. Raska. 2017. Alternative intronic promoters in development and disease. Protoplasma 254: 1201-1206; Kim, H. K., M. H. C. Pham, K. S. Ko, B. D. Rhee, and J. Han. 2018. Alternative splicing isoforms in health and disease. Pflugers Arch. 470: 995-1016, both incorporated herein by reference). Aberrant splicing or deregulated isoform expression/function can lead to diseases, such as cancer and cardiovascular and metabolic diseases (Dlamini, Z., F. Mokoena, and R. Hull. 2017. Abnormalities in alternative splicing in diabetes: therapeutic targets. J. Mol. Endocrinol. 59: R93-R107, incorporated herein by reference). Recent efforts have been made to investigate deregulated alternative splicing that could be used as diagnostic markers or therapeutic targets for diseases.

Described herein is a novel short isoform of human FOXO3, which has been termed Isoform2, in contrast to the full-length isoform1. While available databases support the presence of a putative FOXO3 isoform2 in human cells and tissues, such as fibroblasts and skeletal muscles, in physiological conditions (found at gtexportal.org/home/transcriptPage), to the inventors knowledge, this isoform has never been cloned or characterized. Further, the biological function of this FOXO3 isoform2 was previously unknown.

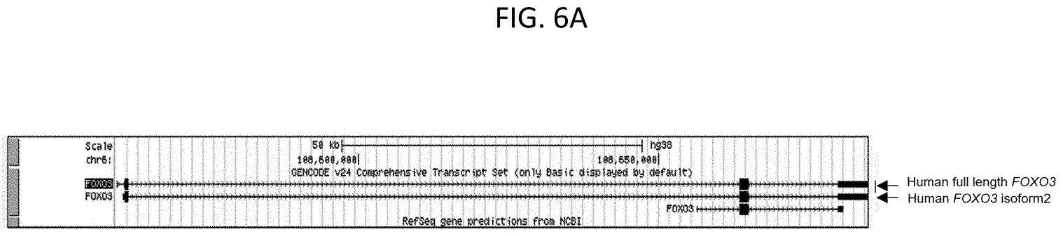

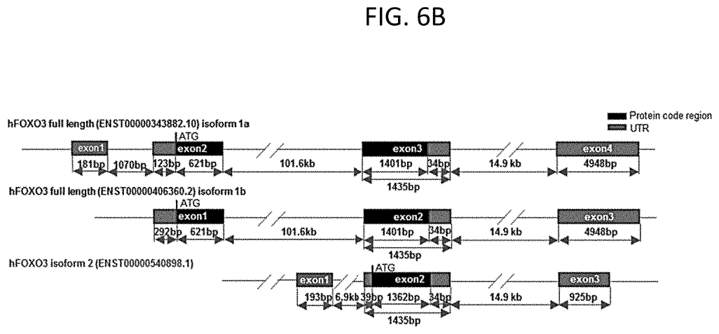

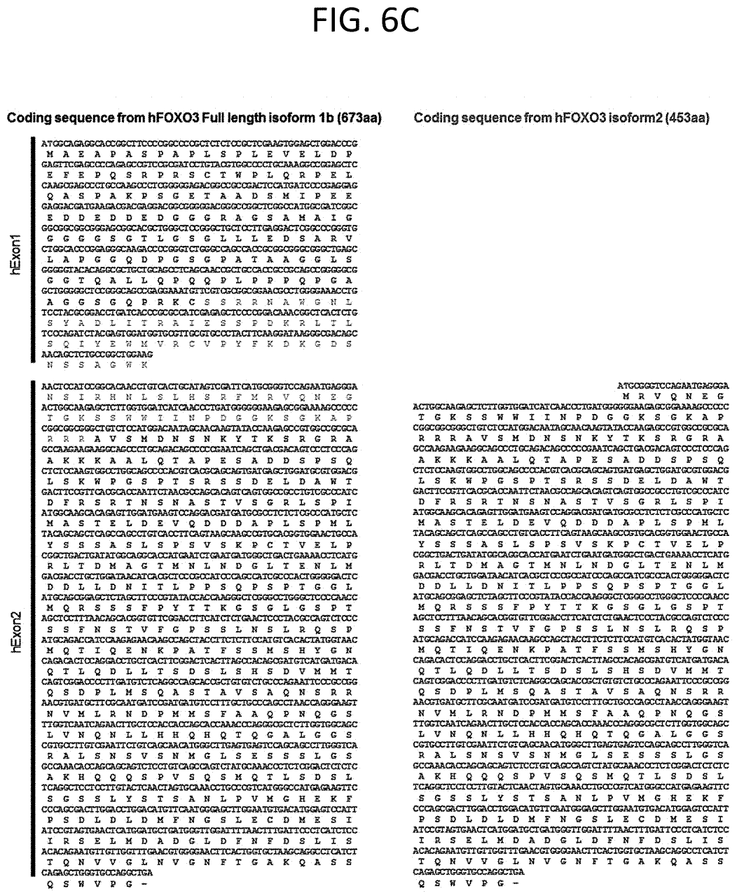

When the inventors investigated the human FOXO3 locus, annotations for a short isoform of FOXO3 (FIG. 6A) were found, which is named as isoform2 (RefSeq gene database, Ensembl genome database, and Uniprot Knowledgebase). The full length of hFOXO3 is named as isoform1, which contains 673 aa. The human full-length FOXO3 isoform1 has two subisoforms (1a and 1b), which have an identical coding sequence with variable 59 untranslated region. The isoform2, generated by alternative splicing with an alternate promoter, is a truncated FOXO3 protein with 453 aa that are encoded by exon 2 (FIG. 6B, 6C). The amino acid sequence of Foxo3 isoform 2 is set forth in

| SEQ ID NO: 1: | |
|---|---|
| MRVQNEGTGK SSWWIINPDG GKSGKAPRRR AVSMDNSNKY | |
| TKSRGRAAKK KAALQTAPES ADDSPSQLSK WPGSPTSRSS DELDAWTDFR | |
| SRTNSNASTV SGRLSPIMAS TELDEVQDDD APLSPMLYSS SASLSPSVSK | |
| PCTVELPRLT DMAGTMNLND GLTENLMDDL LDNITLPPSQ PSPTGGLMQR | |
| SSSFPYTTKG SGLGSPTSSF NSTVFGPSSL NSLRQSPMQT IQENKPATFS | |
| SMSHYGNQTL QDLLTSDSLS HSDVMMTQSD PLMSQASTAV AQNSRRNVM | |
| LRNDPMMSFA AQPNQGSLVN QNLLHHQHQT QGALGGSRAL | |
| SNSVSNMGLS ESSSLGSAKH QQQSPVSQSM QTLSDSLSGS SLYSTSANLP | |
| VMGHEKFPSD LDLDMFNGSL ECDMESIIRS ELMDADGLDF | |
| NFDSLISTQN VVGLNVGNFT GAKQASSQSW VPG | |
| The coding sequence is set forth in SEQ ID NO: 2: | |
| atgcgggtcc agaatgaggg aactggcaag agctcttggt ggatcatcaa ccctgatggg | 60 |
| gggaagagcg gaaaagcccc ccggcggcgg gctgtctcca tggacaatag caacaagtat | 120 |
| accaagagcc gtggccgcgc agccaagaag aaggcagccc tgcagacagc ccccgaatca | 180 |
| gctgacgaca gtccctccca gctctccaag tggcctggca gccccacgtc acgcagcagt | 240 |
| gatgagctgg atgcgtggac ggacttccgt tcacgcacca attctaacgc cagcacagtc | 300 |
| agtggccgcc tgtcgcccat catggcaagc acagagttgg atgaagtcca ggacgatgat | 360 |
| gcgcctctct cgcccatgct ctacagcagc tcagccagcc tgtcaccttc agtaagcaag | 420 |
| ccgtgcacgg tggaactgcc acggctgact gatatggcag gcaccatgaa tctgaatgat | 480 |
| gggctgactg aaaacctcat ggacgacctg ctggataaca tcacgctccc gccatcccag | 540 |
| ccatcgccca ctgggggact catgcagcgg agctctagct tcccgtatac caccaagggc | 600 |
| tcgggcctgg gctccccaac cagctccttt aacagcacgg tgttcggacc ttcatctctg | 660 |
| aactccctac gccagtctcc catgcagacc atccaagaga acaagccagc taccttctct | 720 |
| tccatgtcac actatggtaa ccagacactc caggacctgc tcacttcgga ctcacttagc | 780 |
| cacagcgatg tcatgatgac acagtcggac cccttgatgt ctcaggccag caccgctgtg | 840 |
| tctgcccaga attcccgccg gaacgtgatg cttcgcaatg atccgatgat gtcctttgct | 900 |
| gcccagccta accagggaag tttggtcaat cagaacttgc tccaccacca gcaccaaacc | 960 |
| cagggcgctc ttggtggcag ccgtgccttg tcgaattctg tcagcaacat gggcttgagt | 1020 |
| gagtccagca gccttgggtc agccaaacac cagcagcagt ctcctgtcag ccagtctatg | 1080 |
| caaaccctct cggactctct ctcaggctcc tccttgtact caactagtgc aaacctgccc | 1140 |
| gtcatgggcc atgagaagtt ccccagcgac ttggacctgg acatgttcaa tgggagcttg | 1200 |
| gaatgtgaca tggagtccat tatccgtagt gaactcatgg atgctgatgg gttggatttt | 1260 |
| aactttgatt ccctcatctc cacacagaat gttgttggtt tgaacgtggg gaacttcact | 1320 |
| ggtgctaagc aggcctcatc tcagagctgg gtgccaggct ga | 1362 |

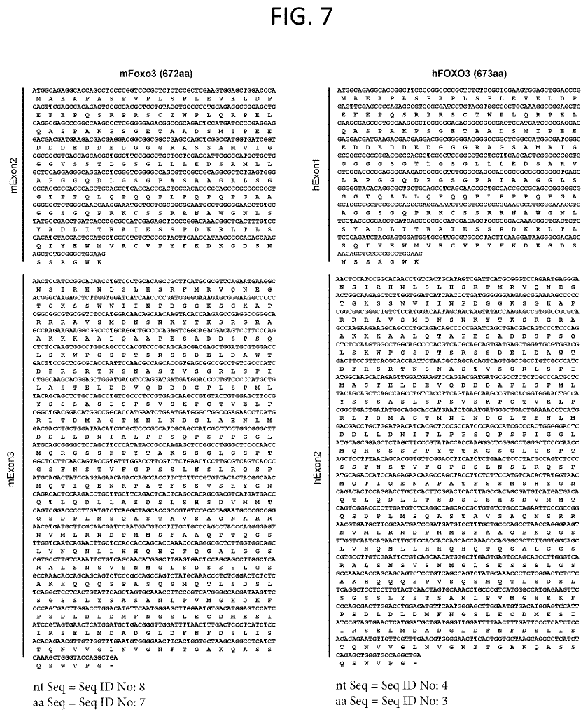

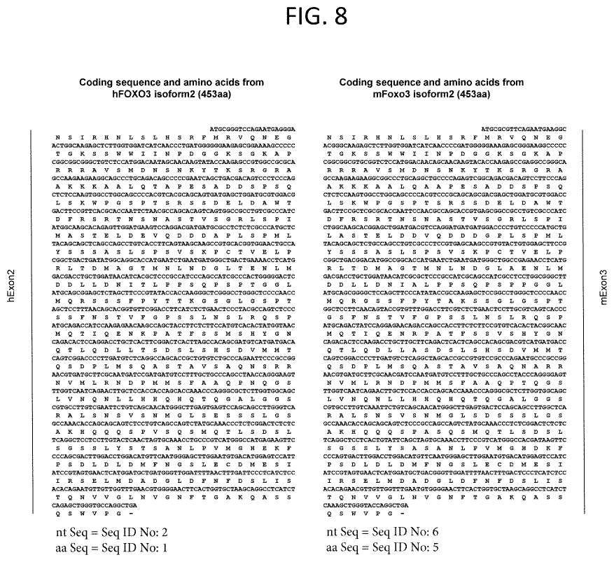

The coding sequences of the mouse and human FOXO3 are highly conserved, demonstrating about 95% identical amino acids (FIG. 7). When comparing the coding and amino acid sequences of the human FOXO3 isoform2 with the mouse truncated Foxo3 in Foxo3^{flox/flox}; LysMcre⁺ BMMs, it was found that 96% of the amino acids are identical (FIG. 8). These new findings indicate that the mouse truncated Foxo3 in Foxo3^{flox/flox}; LysMcre⁺ BMMs is a mouse ortholog of human FOXO3 isoform2. As used herein, this novel Foxo3 isoform is termed as mouse Foxo3 isoform2 (mFoxo3 isoform 2).

Provided herein are compositions and methods for suppressing osteoclast differentiation or function and/or bone resorption or destruction in a subject in need thereof. In one embodiment, the method includes increasing the amount, expression, or activity of Foxo3 isoform 2 in the subject. Compositions for doing so are provided.

In one embodiment, Foxo2 isoform 2 is increased in the subject by administering a nucleic acid which comprises a sequence encoding Foxo3 isoform 2. Thus, in one aspect, a nucleic acid which comprises a sequence encoding Foxo3 isoform 2, or functional fragment thereof, is provided, as well as expression cassettes and vectors containing same. In one embodiment, the nucleic acid encodes the polypeptide sequence of SEQ ID NO: 1, or a sequence sharing at least 90% identity with SEQ ID NO: 1. In another embodiment, the sequence encodes a sequence sharing at least 91%, 92%, 93%, 94%, 95%, 96%, 97%, 98%, or 99% identity with SEQ ID NO: 1. In another embodiment, the nucleic acid encodes a functional fragment of Foxo3 isoform 2, such as the sequence of SEQ ID NO: 1, but having a N-terminal truncation. In one embodiment, the Foxo3 isoform 2 polypeptide has a N-terminal truncation of up to 5, 10, 15, 20, 25, 30, 35, or 40 amino acids. In one embodiment, the functional fragment shares at least 90% identity with the portion of SEQ ID NO: 1 for which corresponding residues are present. For clarity, it is meant that Foxo3 isoform 2 truncations which have been substituted in up to about 10% of the residues present as compared to SEQ ID NO: 1 are encompassed herein.

In one embodiment, the coding sequence is the sequence of SEQ ID NO: 2, or a sequence sharing at least 70% identity therewith. In another embodiment, the coding sequence shares at least 75%, 80%, or 90% with SEQ ID NO: 2. In another embodiment, the coding sequence shares at least 91%, 92%, 93%, 94%, 95%, 96%, 97%, 98%, or 99% with SEQ ID NO: 2.

In one embodiment, the nucleic acid which comprises the Foxo3 isoform 2 coding sequence is contained within an expression cassette, which further includes additional sequences, such as regulatory sequences which permit expression of the Foxo3 isoform 2. These control sequences or the regulatory sequences are operably linked to the Foxo3 isoform 2 coding sequence. As used herein, an “expression cassette” refers to a nucleic acid molecule which comprises coding sequences, promoter, and may include other regulatory sequences therefor, which cassette may be engineered into a genetic element and/or packaged into the capsid of a viral vector (e.g., a viral particle). Typically, such an expression cassette for generating a viral vector contains the sequences described herein flanked by packaging signals of the viral genome and other expression control sequences such as those described herein.

The expression cassette typically contains a promoter sequence as part of the expression control sequences or the regulatory sequences. Promoters such as tissue-specific promoters, viral promoters, constitutive promoters, regulatable promoters [see, e.g., WO 2011/126808 and WO 2013/049493], or a promoter responsive to physiologic cues may be utilized in the vectors described herein.

In addition to a promoter, an expression cassette and/or a vector may contain other appropriate “regulatory elements” or “regulatory sequences”, which comprise but are not limited to enhancers; transcription factors; transcription terminators; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., the alpha fetoprotein enhancer, the TTR minimal promoter/enhancer, LSP (TH-binding globulin promoter/alpha1-microglobulin/bikunin enhancer), amongst others.

In one embodiment, the viral vector is an adenoviral vector. Adenoviruses are medium-sized (90-100 nm), nonenveloped (naked) icosahedral viruses composed of a nucleocapsid and a double-stranded linear DNA genome. There are over 51 different serotypes in humans, which are responsible for 5-10% of upper respiratory infections in children, and many infections in adults as well. In one embodiment, the vector is a replication defective adenovirus, in which the E1A and E1B genes are deleted and replaced with an expression cassette comprising the Foxo3 isoform 2 coding sequence. Various adenoviral vectors are known in the art and include, without limitation, Ad5 based vectors. See, e.g., Wold and Toth, Adenovirus Vectors for Gene Therapy, Vaccination and Cancer Gene Therapy, Curr Gene Ther. 2013 December; 13(6): 421-433, which is incorporated herein by reference.

In another embodiment, the viral vector is an adeno-associated virus (AAV) vector. AAV is composed of an icosahedral protein capsid of ˜26 nm in diameter and a single-stranded DNA genome of ˜4.7 kb that can either be the plus (sense) or minus (anti-sense) strand. The capsid comprises three types of subunit, VP1, VP2 and VP3, totaling 60 copies in a ratio of about 1:1:10 (VP1:VP2:VP3). The genome is flanked by two T-shaped inverted terminal repeats (ITRs) at the ends that largely serve as the viral origins of replication and the packaging signal. Various AAV vectors are known in the art and include, without limitation, AAV1, AAV2, AAV3B, AAV4, AAV5, AAV6, AAV7, AAV8, AAV9, AAVrh.8, AAVrh.10 and AAVrh.43 based vectors. See, e.g., Wang et al, Adeno-associated virus vector as a platform for gene therapy delivery, Nature Reviews Drug Discovery, 18: 358-378 (February 2019), which is incorporated herein by reference.

In another embodiment of the methods provided herein, Foxo2 isoform 2 is increased in the subject by administering an effective amount of Foxo3 isoform 2 polypeptide. Thus, in one embodiment, a composition comprising a Foxo3 isoform 2 polypeptide is provided. In one embodiment, the polypeptide has the sequence of SEQ ID NO: 1, or a sequence sharing at least 90% identity with SEQ ID NO: 1. In another embodiment, the sequence encodes a sequence sharing at least 91%, 92%, 93%, 94%, 95%, 96%, 97%, 98%, or 99% identity with SEQ ID NO: 1. In another embodiment, the polypeptide is a functional fragment of Foxo3 isoform 2. In one embodiment, the Foxo3 isoform 2 fragment polypeptide has a N-terminal truncation of up to 5, 10, 15, 20, 25, 30, 35, or 40 amino acids. In one embodiment, the functional fragment shares at least 90% identity with the portion of SEQ ID NO: 1 for which corresponding residues are present. For clarity, it is meant that Foxo3 isoform 2 truncations which have been substituted in up to about 10% of the residues present are encompassed herein.

The “effective amount” for of a Foxo3 isoform 2 polypeptide can be about 0.01 to 25 mg peptide per application. In one embodiment, the effective amount is 0.01 to 10 mg. In another embodiment, the effective amount is 0.01 to 1 mg. In another embodiment, the effective amount is 0.01 to 0.10. In another embodiment, the effective amount is 0.2, 0.5, 0.8, 1.0, 1.2, 1.4, 1.6, 1.8, 2.0, 2.2, 2.4, 2.6, 2.8, 3.0 mg or more.

In another embodiment, Foxo3 isoform 2 is increased in the subject by administering an effective amount of a Foxo3 isoform 2 agonist. In one embodiment, the effective amount of the Foxo3 isoform 2 agonist is an amount ranging from about 0.01 mg/ml to about 10 mg/ml, including all amounts therebetween and end points. In one embodiment, the effective amount of the Foxo3 isoform 2 agonist is about 0.1 mg/ml to about 5 mg/ml, including all amounts therebetween and end points. In another embodiment, the effective amount of the Foxo3 isoform 2 agonist is about 0.3 mg/ml to about 1.0 mg/ml, including all amounts therebetween and end points. In another embodiment, the effective amount of the Foxo3 isoform 2 agonist is about 0.3 mg/ml. In another embodiment, the effective amount of the Foxo3 isoform 2 agonist is about 0.4 mg/ml. In another embodiment, the effective amount of the Foxo3 isoform 2 agonist is about 0.5 mg/ml. In another embodiment, the effective amount of the Foxo3 isoform 2 agonist is about 0.6 mg/ml. In another embodiment, the effective amount of the Foxo3 isoform 2 agonist is about 0.7 mg/ml. In another embodiment, the effective amount of the Foxo3 isoform 2 agonist is about 0.8 mg/ml. In another embodiment, the effective amount of the Foxo3 isoform 2 agonist is about 0.9 mg/ml. In another embodiment, the effective amount of the Foxo3 isoform 2 agonist is about 1.0 mg/ml.

In one embodiment, the effective amount of the Foxo3 isoform 2 agonist is an amount ranging from about 1 μM to about 2 mM, including all amounts therebetween and end points. In one embodiment, the effective amount of the Foxo3 isoform 2 agonist is about 10 μM to about 100 μM, including all amounts therebetween and end points. In another embodiment, the effective amount of the Foxo3 isoform 2 agonist is about 5 μM. In another embodiment, the effective amount of the Foxo3 isoform 2 agonist is about 10 μM. In another embodiment, the effective amount of the Foxo3 isoform 2 agonist is about 20 μM. In another embodiment, the effective amount of the Foxo3 isoform 2 agonist is about 50 μM. In another embodiment, the effective amount of the Foxo3 isoform 2 agonist is about 100 μM. In another embodiment, the effective amount of the Foxo3 isoform 2 agonist is about 200 μM. In another embodiment, the effective amount of the Foxo3 isoform 2 agonist is about 300 μM. In another embodiment, the effective amount of the Foxo3 isoform 2 agonist is about 400 μM. In another embodiment, the effective amount of the Foxo3 isoform 2 agonist is about 500 μM. In another embodiment, the effective amount of the Foxo3 isoform 2 agonist is about 600 μM. In another embodiment, the effective amount of the Foxo3 isoform 2 agonist is about 700 μM. In another embodiment, the effective amount of the Foxo3 isoform 2 agonist is about 800 μM. In another embodiment, the effective amount of the Foxo3 isoform 2 agonist is about 900 μM. In another embodiment, the effective amount of the Foxo3 isoform 2 agonist is about 1 mM. In another embodiment, the effective amount of the Foxo3 isoform 2 agonist is about 1.25 mM. In another embodiment, the effective amount of the Foxo3 isoform 2 agonist about 1.5 mM. In another embodiment, the effective amount of the Foxo3 isoform 2 agonist is about 1.75 mM. In another embodiment, the effective amount of the Foxo3 isoform 2 agonist is about 2 mM.

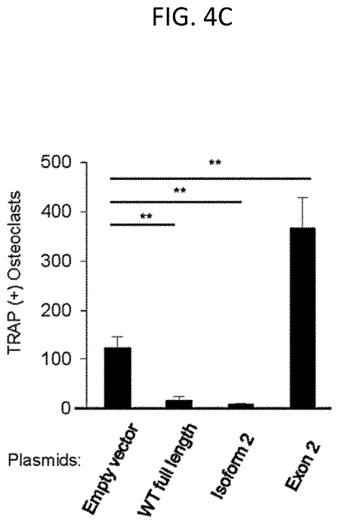

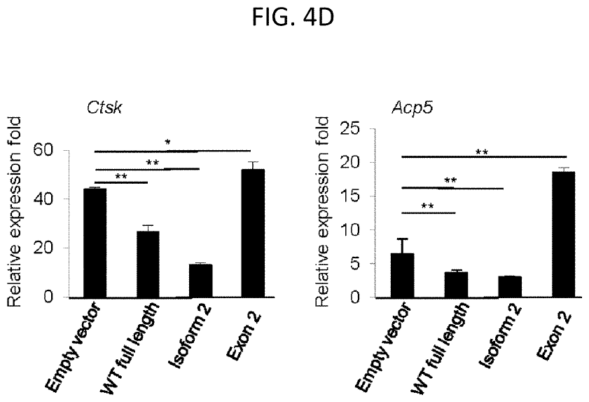

As shown in FIG. 7, human Foxo3 exon 1 aligns with mouse Foxo3 exon 2. Whereas in the mouse Foxo3 isoform 2, the coding sequence begins in mExon3, in human, the coding sequence begins in hExon 2. As shown in FIG. 4C, supplementing mExon 2 in RAW264.7 cells increased osteoclastogenesis. Thus, in one embodiment, a method for suppressing osteoclast differentiation or function and/or bone resorption or destruction in a subject in need thereof includes disrupting hExon 1. In one embodiment, hExon 1 is disrupted via s small molecule which binds or interferes with the structure of hExon 1.

For each of the nucleic acids, polypeptide, and agonist compositions described herein, a further embodiment is provided which additionally includes a pharmaceutically acceptable carrier. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

Routes of administration include, but are not limited to, intradermal, intramuscular, intraperitoneal, intravenous, subcutaneous, intranasal, epidural, and oral routes. The agent may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In some embodiments, the methods of treatment include combination with another therapy. Such additional therapies include without limitation, nonsteroidal anti-inflammatory drugs (NSAIDs), steroids such as prednisone, methotrexate (Trexall, Otrexup, others), leflunomide (Arava), hydroxychloroquine (Plaquenil) and sulfasalazine (Azulfidine), abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) and tofacitinib (Xeljanz). Other additional therapies include Bisphosphonates including Alendronate (Fosamax), Risedronate (Actonel), Ibandronate (Boniva), and Zoledronic acid (Reclast). Other therapies include hormone like medications including raloxifene (Evista), Denosumab (Prolia, Xgeva), Teriparatide (Forteo), Abaloparatide (Tymlos).

As described herein, it has been shown that expression of Foxo3 isoform 2 suppresses osteoclastogenesis. Thus, in one method is provided a method of suppressing osteoclastogenesis or osteoclast differentiation or function in a subject in need thereof. The method includes increasing the expression, amount or activity of Foxo3 isoform 2, as further described herein. In another embodiment, a method of suppressing or decreasing bone resorption or destruction in a subject in need thereof is provided. The method includes increasing the expression, amount or activity of Foxo3 isoform 2, as further described herein. In yet another embodiment, a method of treating a skeletal disease is provided. The method includes increasing the expression, amount or activity of Foxo3 isoform 2, as further described herein.

In any of the methods described herein, the subject may have, or be suspected of having or developing, a skeletal disease, as described hereinabove. In one embodiment, the subject has, or is suspected of having or developing, rheumatoid arthritis. In another embodiment, the subject has, or is suspected of having or developing, psoriatic arthritis. In another embodiment, the subject has, or is suspected of having or developing, periodontitis. In another embodiment, the subject has, or is suspected of having or developing, periprosthetic loosening. In another embodiment, the subject has, or is suspected of having or developing, osteoporosis.

In another aspect, a method of diagnosing an increased risk of developing a skeletal disease is provided. The method includes measuring the level of Foxo3 isoform 2 in a sample from a subject. In one embodiment, the sample is whole blood. In another embodiment, the sample is PBMC. In some embodiments, the level of Foxo3 isoform 2 is detected in a sample obtained from a subject. This level may be compared to the level of a control. In one embodiment, a decrease in the level of Foxo3 isoform 2 as compared to a control indicates a greater risk of developing a skeletal disease. In one embodiment, a level of 100 ng/mL or lower is indicative of an increased risk of a skeletal disease in the subject, as compared to a control. “Control” or “control level” as used herein refers to the source of the reference value for Foxo3 isoform 2 levels. In some embodiments, the control subject is a healthy subject with no disease. In yet other embodiments, the control or reference is the same subject from an earlier time point. Selection of the particular class of controls depends upon the use to which the diagnostic/monitoring methods and compositions are to be put by the care provider. The control may be a single subject or population, or the value derived therefrom.

In another aspect, a method of diagnosing a skeletal disease in a subject is provided. The method includes measuring the level of Foxo3 isoform 2 a sample from a subject. In one embodiment, the sample is whole blood. In another embodiment, the sample is PBMC. This level may be compared to the level of a control. In one embodiment, a decrease in the level of Foxo3 isoform 2 as compared to a control indicates the presence of a skeletal disease. In one embodiment, a level of 1 ng/mL or lower is indicative of the presence of a skeletal disease in the subject. “Control” or “control level” as used herein refers to the source of the reference value for Foxo3 isoform 2 levels. In some embodiments, the control subject is a healthy subject with no disease. In yet other embodiments, the control or reference is the same subject from an earlier time point. Selection of the particular class of controls depends upon the use to which the diagnostic/monitoring methods and compositions are to be put by the care provider. The control may be a single subject or population, or the value derived therefrom. In one embodiment, the method further includes treating the subject for the skeletal disease. In one embodiment, the treatment is selected from a nonsteroidal anti-inflammatory drug (NSAID), a steroid such as prednisone, methotrexate (Trexall, Otrexup, others), leflunomide (Arava), hydroxychloroquine (Plaquenil) and sulfasalazine (Azulfidine), abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) and tofacitinib (Xeljanz). In one embodiment, the treatment is a Bisphosphonate selected from Alendronate (Fosamax), Risedronate (Actonel), Ibandronate (Boniva), and Zoledronic acid (Reclast). In another embodiment, the treatment is raloxifene (Evista), Denosumab (Prolia, Xgeva), Teriparatide (Forteo), or Abaloparatide (Tymlos). In one embodiment, the subject is treated by increasing the Foxo3 isoform 2, as described herein.

In another aspect, a method of assessing the efficacy of a treatment for a skeletal disease is provided. In one embodiment, a baseline level of Foxo3 isoform 2 is obtained from the subject prior to, or at the beginning of treatment for a skeletal disease. After a desirable time period, the level of Foxo3 isoform 2 in the subject is measured again. An increase in the level of Foxo3 isoform 2 as compared to the earlier time point indicates that the treatment for the skeletal disease is, at least partially, efficacious. The treatment may be any of those described herein, or other treatments deemed suitable by the health care provider.

In another aspect, a method of screening for a compound useful for treating a skeletal disease is provided. In one embodiment, the compound is administered to a Foxo3^{f/f};LysMcre (Foxo3^{isoform2}) mouse. In one embodiment, a baseline level of Foxo3 isoform 2 is obtained from the mouse prior to, or at the beginning of testing. After a desirable time period, the level of Foxo3 isoform 2 in the mouse is measured again. An increase in the level of Foxo3 isoform 2 as compared to the earlier time point indicates that the compound is, at least partially, efficacious for treatment of a skeletal disease.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not intended to limit the present invention.

### EXAMPLES

### Example 1: Materials and Methods

### Plasmids, Cloning, and Sequencing

cDNA fragments encoding mouse full-length Foxo3 protein or exon 2 fused with FLAG tag at the C terminus was amplified by PCR using the cDNA templates from WT BMMs and then subcloned into the Xba1I/BamHI sites of pcDNA3.1+ vector to construct the pcDNA3.1+ full-length Foxo3-Flag plasmid or pcDNA3.1+-Foxo3 exon 2-Flag plasmid, respectively. Furthermore, cDNA fragment encoding mouse Foxo3 isoform2 fused with FLAG tag at the C terminus was amplified by PCR using the cDNA templates from Foxo3^{isoform2} BMMs, followed by subcloning into the Xba1I/BamHI sites of pcDNA3.1+ vector to construct the pcDNA3.1⁺-Foxo3 isoform2-Flag plasmid. The following primers were used for cloning: for Foxo3 full-length fragment,

forward
(SEQ ID NO: 9)
5′-ATTCTAGAGCCACCATGGCAGAGGCACCAGCC-3′,
reverse
(SEQ ID NO: 10)
5′-ATGGATCCTCACTTGTCGTCATCGTCTTTGTAGTCGCCTGGTACCCAG
CTTTGA-3′;
for exon 2 of Foxo3 fragment,
forward
(SEQ ID NO: 11)
5′-ATTCTAGAGCCACCATGGCAGAGGCACCAGCC-3′,
reverse
(SEQ ID NO: 12)
5′-ATGGATCCTCACTTGTCGTCATCGTCTTTGTAGTCCTTCCAGCCCGCA
GAGCT-3′;
and
for Foxo3 isoform2 fragment,
forward
(SEQ ID NO: 13)
5′-ATTCTAGAGCCACCATGCGCGTTCAGAATGAAGG-3′,
reverse
(SEQ ID NO: 14)
5′-ATGGATCCTCACTTGTCGTCATCGTCTTTGTAGTCGCCTGGTACCCAG
CTTTGA-3′.

The sequence integrity of the inserted fragments in each expression plasmid was verified by restriction enzyme digestion and DNA sequencing at Cornell University Genomics Facility.

### Transfection of Human Embryonic Kidney 293 Cells and RAW264.7 Cells

Lipofectamine 3000 reagent (L3000015; Thermo Fisher Scientific) was used for the transfection of the human embryonic kidney (HEK) 293 cells or RAW264.7 cells. Briefly, the cells were seeded (2.5×10⁵ HEK cells/well and 1.2×10⁵ RAW264.7 cells/well) and cultured with DMEM for HEK293 cells or a-MEM for RAW264.7 cells supplemented with 10% FBS and 1% penicillin/streptomycin in a 24-well plate at 37° C. in a humidified atmosphere containing 5% CO2 overnight. The cells were then transfected with 500 ng plasmid DNAs using Lipofectamine 3000 reagent, according to the manufacturer's instructions. After 24 h, the medium was replaced with fresh completed DMEM for HEK293 cells or a-MEM for RAW264.7 cells. The protein lysates from cell cultures were collected after 48 h to assess plasmid expression.

### In Vitro Gene Silencing by Small Interfering RNAs

In vitro gene silencing by small interfering RNAs (siRNAs) was performed as previously described (Miller, C. H., S. M. Smith, M. Elguindy, T. Zhang, J. Z. Xiang, X. Hu, L. B. Ivashkiv, and B. Zhao. 2016. RBP-J-Regulated miR-182 Promotes TNF-alpha-Induced Osteoclastogenesis. Journal of immunology 196: 4977-4986.). Briefly, siRNAs targeting Foxo3 or their corresponding control oligos (80 nM) were transfected into murine BMMs using TransIT-TKO transfection reagent (Mirus Bio), in accordance with the manufacturer's instructions.

### RNA Sequencing and Bioinformatics Analysis

RNA sequencing (RNA-seq) and bioinformatics analysis were performed as previously described (Inoue, K., Z. Deng, Y. Chen, E. Giannopoulou, R. Xu, S. Gong, M. B. Greenblatt, L. S. Mangala, G. Lopez-Berestein, D. G. Kirsch, et al. 2018. Bone protection by inhibition of microRNA-182. Nat. Commun. 9: 4108.). Briefly, total RNAwas extracted using RNeasy Mini Kit (QIAGEN) following the manufacturer's instructions. TruSeq RNA Library preparation kits (Illumina) were used to purify poly-A+ transcripts and generate libraries with multiplexed barcode adaptors, following the manufacturer's instructions. All samples passed quality control analysis using a Bioanalyzer 2100 (Agilent Technologies). RNA-seq libraries were constructed per the Illumina TruSeq RNA sample preparation kit. High throughput sequencing was performed using the Illumina HiSeq 4000 in the Weill Cornell Medical College Genomics Resources Core Facility. RNAseq reads were aligned to the mouse genome (mm10) using TopHat (Trapnell, C., L. Pachter, and S. L. Salzberg. 2009. TopHat: discovering splice junctions with RNA-Seq. Bioinformatics 25: 1105-1111.). Cufflinks (Trapnell, C., B. A. Williams, G. Pertea, A. Mortazavi, G. Kwan, M. J. van Baren, S. L. Salzberg, B. J. Wold, and L. Pachter. 2010. Transcript assembly and quantification by RNA-Seq reveals unannotated transcripts and isoform switching during cell differentiation. Nat. Biotechnol. 28: 511-515.) was subsequently used to assemble the aligned reads into transcripts and then estimate the transcript abundances as reads per kilo base per million values. HTseq (Anders, S., P. T. Pyl, and W. Huber. 2015. HTSeq—a Python framework to work with high-throughput sequencing data. Bioinformatics 31: 166-169.) was used to calculate raw reads counts, and edgeR (Robinson, M. D., D. J. McCarthy, and G. K. Smyth. 2010. edgeR: a Bioconductor package for differential expression analysis of digital gene expression data. Bioinformatics 26: 139-140.) was used to calculate normalized counts as counts per million.

Heatmaps were generated by pheatmap package in R. RNA-seq data (accession no. GSE 135479) have been deposited in National Center for Biotechnology Information's Gene Expression Omnibus (http://www ncbi.nlm nih.gov/geo/query/acc.cgi?acc=GSE 135479).

### Reverse Transcription and Real-Time PCR

Reverse transcription and real-time PCR were performed as previously described (Inoue, K., Z. Deng, Y. Chen, E. Giannopoulou, R. Xu, S. Gong, M. B. Greenblatt, L. S. Mangala, G. Lopez-Berestein, D. G. Kirsch, et al. 2018. Bone protection by inhibition of microRNA-182. Nat. Commun. 9: 4108.). DNA-free RNA was obtained with the RNeasy Mini Kit (no. 74106; QIAGEN, Valencia, Calif.) with DNase treatment, and 1 mg of total RNAwas reverse transcribed using a First Strand cDNA Synthesis Kit (Thermo Fisher Scientific, Waltham, Mass.), according to the manufacturer's instructions. Real-time PCR was done in triplicate with the QuantStudio 5 Real-time PCR System and Fast SYBR Green Master Mix (Thermo Fisher Scientific). Gene expression was normalized relative to GAPDH. The primers for real-time PCR were as follows:

Acp5:
SEQ ID NO: 15
5′-ACGGCTACTTGCGGTTTC-3′
and
SEQ ID NO: 16
5′-TCCTTGGGAGGCTGGTC-3′;
Dcstamp:
SEQ ID NO: 17
5′-TTTGCCGCTGTGGACTATCTGC-3′
and
SEQ ID NO: 18
5′-AGACGTGGTTTAGGAATGCAGCTC-3′;
Ctsk:
SEQ ID NO: 19
5′-AAGATATTGGTGGCTTTGG-3′
and
SEQ ID NO: 20
5′-ATCGCTGCGTCCCTCT-3′;
Itgb3:
SEQ ID NO: 21
5′-CCGGGGGACTTAATGAGACCACTT-3′
and
SEQ ID NO: 22
5′-ACGCCCCAAATCCCACCCATACA-3′;
Calcr:
SEQ ID NO: 23
5′-ACATGATCCAGTTCACCAGGCAGA-3′
and
SEQ ID NO: 24
5′-AGGTTCTTGGTGACCTCCCAACTT-3′;
Foxo3-F3R3:
SEQ ID NO: 25
5′-CTGTCCTATGCCGACCTGAT-3′
and
SEQ ID NO: 26
5′-CTGTCGCCCTTATCCTTGAA-3′;
Foxo3-F4R4:
SEQ ID NO: 27
5′-ATGGGAGCTTGGAATGTGAC-3′
and
SEQ ID NO: 28
5′-TTAAAATCCAACCCGTCAGC-3′;
Foxo3-F5R5:
SEQ ID NO: 29
5′-AGGAGGAGGAATGTGGAAGG-3′
and
SEQ ID NO: 30
5′-CCGTGCCTTCATTCTGAAC-3′;
Ifnb1:
SEQ ID NO: 31
5′-TTACACTGCCTTTGCCATCC-3′
and
SEQ ID NO: 32
5′-AGAAACACTGTCTGCTGGTG-3′;
Mx1:
SEQ ID NO: 33
5′-GGCAGACACCACATACAACC-3′
and
SEQ ID NO: 34
5′-CCTCAGGCTAGATGGCAAG-3′;
Ifit1:
SEQ ID NO: 35
5′-CTCCACTTTCAGAGCCTTCG-3′
and
SEQ ID NO: 36
5′-TGCTGAGATGGACTGTGAGG-3′;
Irf7:
SEQ ID NO: 37
5′-GTCTCGGCTTGTGCTTGTCT-3′
and
SEQ ID NO: 38
5′-CCAGGTCCATGAGGAAGTGT-3′;
Ifit2:
SEQ ID NO: 39
5′-AAATGTCATGGGTACTGGAGTT-3′
and
SEQ ID NO: 40
5′-ATGGCAATTATCAAGTTTGTGG-3′;
Stat1:
SEQ ID NO: 41
5′-CAGATATTATTCGCAACTACAA-3′
and
SEQ ID NO: 42
5′-TGGGGTACAGATACTTCAGG-3′;
and
Gapdh:
SEQ ID NO: 43
5′-ATCAAGAAGGTGGTGAAGCA-3′
and
SEQ ID NO: 44
5′-AGACAACCTGGTCCTCAGTGT-3′.

### Immunoblot Analysis

Total cell extracts were obtained using lysis buffer containing 150mMTris-HCl (pH 6.8), 6% SDS, 30% glycerol, and 0.03% bromophenol blue; 10% 2-ME was added immediately before harvesting cells. Cell lysates were fractionated on SDS-PAGE, transferred to Immobilon-P membranes (MilliporeSigma), and incubated with specific Abs. Western Lightning Plus-ECL (PerkinElmer) was used for detection. Foxo3 N-terminal (no. 2497, specifically recognizing the residues surrounding Glu50 in exon 2 of Foxo3) and C-terminal (no. 12829S, specifically recognizing the C terminus of Foxo3) Abs were purchased from Cell Signaling Technology. Anti-Flag tag Ab (no. 637301) was purchased from BioLegend. p38a (sc-535) Ab was from Santa Cruz Biotechnology.

### Statistical Analysis

Statistical analysis was performed using GraphPad Prism software. Two-tailed Student t test was applied if there were only two groups of samples. In the case of more than two groups of samples, one-way ANOVA was used with one condition, and two-way ANOVA was used with more than two conditions. ANOVA analysis was followed by post hoc Bonferroni correction for multiple comparisons. A p value <0.05 was taken as statistically significant: *p<0.05 and **p<0.01. Data are presented as the mean±SD, as indicated in the figure legends.

### Example 2: Results

### Absence of Foxo3 Enhances Osteoclastogenesis

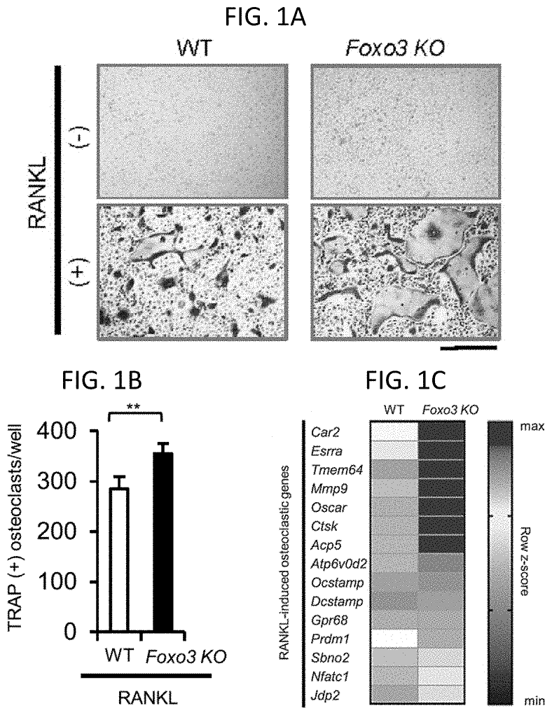

To provide genetic evidence for the role of Foxo3 in osteoclasts, we first took advantage of Foxo3 global KO mice, in which the Foxo3 protein is completely deleted. We first used BMMs as osteoclast precursors to examine in vitro osteoclast differentiation in response to RANKL, the master osteoclastogenic inducer. We found that Foxo3 KO-derived BMMs showed an increased responsiveness to RANKL, determined by more TRAP-positive multinucleated osteoclasts (FIG. 1A, 1B). Furthermore, we performed an RNA-seq experiment using WT and Foxo3 KO BMMs to examine gene expression in response to RANKL. In parallel with increased osteoclast formation, the expression of osteoclastic genes, such as Nfatcl (encoding NFATc1), Prdm1 (encoding Blimp1), Acp (encoding TRAP), Oscar (encoding OSCAR), and Ctsk (encoding cathepsin K), was significantly enhanced by RANKL in Foxo3 KO BMM cultures compared with the BMMs cultured from WT controls (FIG. 1C). These results indicate that Foxo3 functions as a negative regulator in RANKL-induced osteoclast differentiation.

Foxo3^{f/f};LysMcre Mice Express a Truncated Foxo3 Protein that is an Ortholog of Human FOXO3 Isoform2.

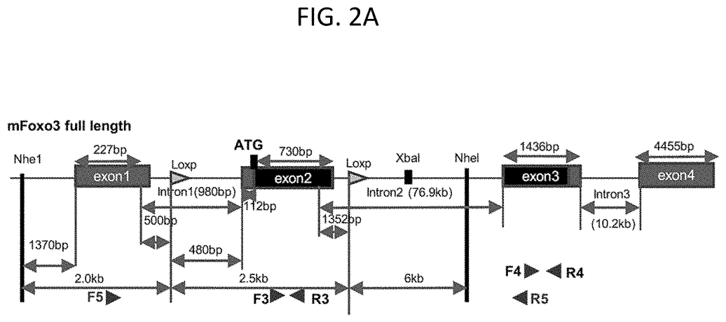

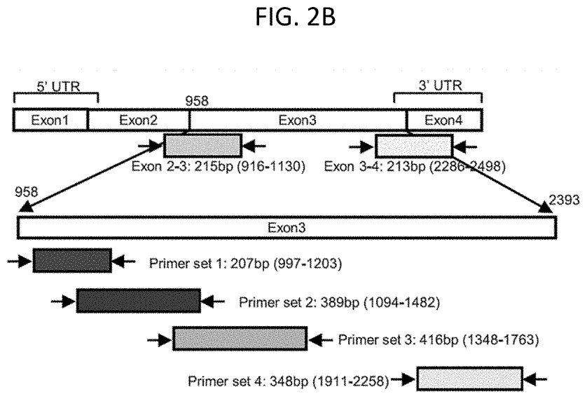

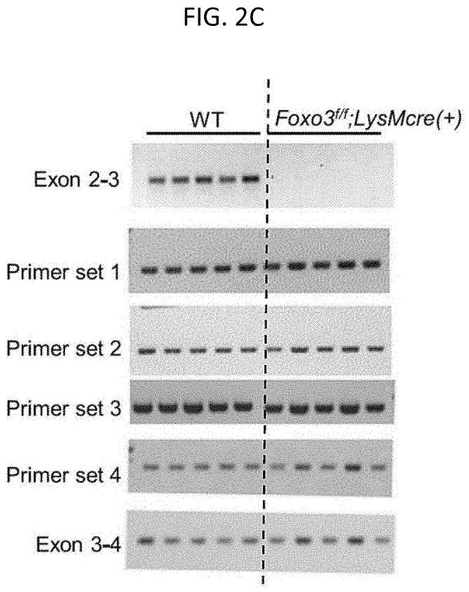

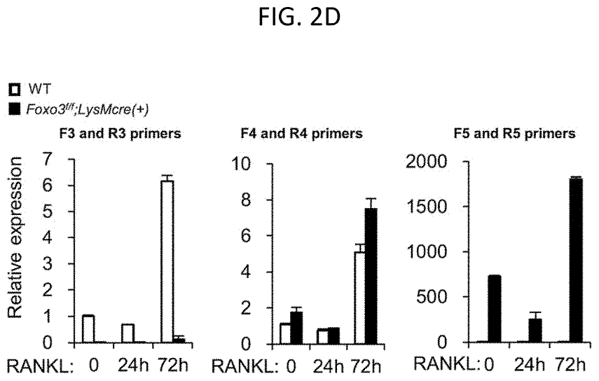

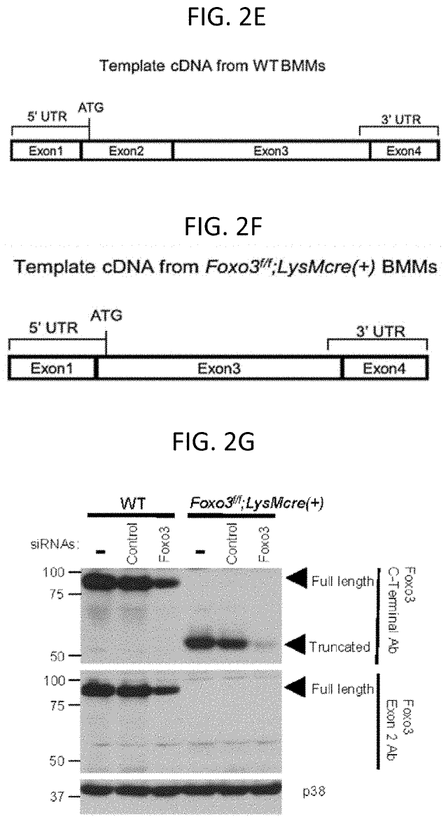

We next wished to examine the role of Foxo3 in vivo using conditional Foxo3 KO mice. We deleted Foxo3 (encoding Foxo3) in myeloid lineage osteoclast precursors by crossing Foxo3flox/flox mice (The Jackson Laboratory) with LysMcre mice that express Cre under the control of the myeloid-specific lysozyme M promoter. We used Foxo3^{flox/flox}; LysMcre+ mice and littermate controls with a Foxo3^{+/+}; LysMcre⁺ genotype (hereafter referred to as WT) in the experiments. The mouse Foxo3 gene has four exons, and the coding region within exons 2 and 3 produces a full-length Foxo3 protein with 672 aa. The Foxo3^{flox/flox} mice (The Jackson Laboratory) possess 1oxP sites flanking exon 2 of the Foxo3 gene (FIG. 2A). To verify Foxo3 deletion, we first designed a series of PCR primers that cover the coding region from exon 2 and exon 3 (FIG. 2B, Table I).

TABLE I
Sequences of regular PCR Primers

| | | Product | |
|---|---|---|---|
| | | Size | SEQ ID |
| Primer Name | Sequences | (bp) | NO |
| Exons 2-3 | F: 5′-TTCAAGGATAAGGGCGACAG-3′ | 215 | 45 |
| | R: 5′-CCTCGGCTCTTGGTGTACTT-3′ | | 46 |
| Exons 3-4 | F: 5′-CGTTGTTGGTTTGAATGTGG-3′ | 213 | 47 |
| | R: 5′-CGTGGGAGTCTCAAAGGTGT-3′ | | 48 |
| Primer Set 1 | F: 5′-ATGCGCGTTCAGAATGAAG-3′ | 207 | 49 |
| | R: 5′-GGAGAGCTGGGAAGGACTGT-3′ | | 50 |
| Primer Set 2 | F: 5′-CCATGGACAACAGCAACAAG-3′ | 389 | 51 |
| | R: 5′-CAGCCCATCATTCAGATTCA-3′ | | 52 |
| Primer Set 3 | F: 5′-GATGATGATGGACCCCTGTC-3′ | 416 | 53 |
| | R: 5′-GAAGCAAGCAGGTCTTGGAG-3′ | | 54 |
| Primer Set 4 | F: 5′-GGGGAGTTTGGTCAATCAGA-3′ | 348 | 55 |
| | R: 5′-TTAAAATCCAACCCGTCAGC-3′ | | 56 |
| F3 and R3 | F: 5′-CTGTCCTATGCCGACCTGAT-3′ | 122 | 57 |
| primers | R: 5′-CTGTCGCCCTTATCCTTGAA-3′ | | 58 |
| F4 and R4 | F: 5′-ATGGGAGCTTGGAATGTGAC-3′ | 73 | 59 |
| primers | R: 5′-TTAAAATCCAACCCGTCAGC-3′ | | 60 |
| F5 and R5 | F: 5′-AGGAGGAGGAATGTGGAAGG-3′ | 221 | 61 |
| primers | R: 5′-CCGTGCCTTCATTCTGAAC-3′ | | 62 |
| Exon 1F | F: 5′-ATTCTAGACTAGGTTGAGGCTCCCTGT-3′ | 2355 | 63 |
| Exon 3R | R: 5′-ATTCCGGATCCGCCTGGTACCCAGCTTTGA-3′ | | 64 |

As shown in FIG. 2C, PCR products were detected in WT BMM cDNAs using all primer sets. As expected, the exon 2-3 primer set did not produce any PCR bands using the Foxo3^{flox/flox}; LysMcre⁺ BMM cDNAs. Surprisingly, other primer sets covering exon 3 or exon 3-4 generated the same PCR products using BMM cDNAs obtained from either Foxo3^{flox/flox}; LysMcre⁺ or WT mice (FIG. 2C). We further designed quantitative PCR primer sets and found that the primers other than those located within exon 2 amplified the Foxo3 cDNAs in Foxo3^{flox/flox}; LysMcre⁺ BMMs (FIG. 2D). These results indicate that there exists a truncated Foxo3 mRNA transcript in the Foxo3^{flox/flox}; LysMcre* mice. Interestingly, the primers located within exon 1 and exon 3 (F5 and R5 primers) were also able to generate PCR products shorter than 300 bp, strongly implying that this truncated Foxo3 mRNA is transcribed from exon 1, skips exon 2, and is elongated to exon 3. To directly demonstrate this, we cloned Foxo3 transcripts from WT or Foxo3^{flox/flox}; LysMcre* BMMs using a primer set (Exon 1F and Exon 3R in FIG. 2E, 2F) that covers WT Foxo3 mRNA starting from the transcription start site in exon 1 to the end of the coding sequence in exon 3. As shown in FIG. 2E, we detected the normal junction between exon 1 and exon 2 in WT BMMs (FIG. 2E). However, the entire exon 2 was absent, and a novel exon 1 to exon 3 junction was present in Foxo3^{flox/flox}; LysMcre* BMMs (FIG. 2F). These results confirm the presence of a novel Foxo3 mRNA with exon 2 truncated in the Foxo3^{flox/flox}; LysMcre⁺ BMMs, resulting from an in-frame (nonframeshift) deletion by the cre-lox recombination in these mice. We next set off to detect the Foxo3 protein expression in the WT and Foxo3^{flox/flox}; LysMcre⁺ BMMs. We used two Abs; one Ab recognizes the C-terminal region of Foxo3, whereas the other is an mAb that specifically targets the exon 2 of Foxo3. As shown in FIG. 2G, the full length of WT Foxo3 proteins were detected by both Abs in WT BMMs. In Foxo3^{flox/flox}; LysMcre⁺ BMMs, the full length of Foxo3 proteins were deleted as expected. In contrast, a truncated Foxo3 protein (55 kDa) was detected by the C-terminal Ab in Foxo3^{flox/flox}; LysMcre⁺ BMMs but not by the Ab specifically targeting exon 2. Furthermore, knockdown of Foxo3 by RNA interference completely deleted the truncated protein (55 kDa) in the Foxo3^{flox/flox}; LysMcre⁺ BMMs (FIG. 2G, top panel). Taken together with the cloning data in FIG. 2F, these results demonstrate that the full-length Foxo3 protein is absent, but there exists an exon 2-truncated Foxo3 protein in Foxo3^{flox/flox}; LysMcre⁺ BMMs.

When we investigated the human FOXO3 locus, we found annotations for a short isoform of FOXO3 (FIG. 6A), which is named as isoform2 (RefSeq gene database, Ensembl genome database, and Uniprot Knowledgebase). The full length of FOXO3 is named as isoform1, which contains 673 aa. The human full-length FOXO3 isoform1 has two subisoforms (1a and 1b), which have an identical coding sequence with variable 59 untranslated region. The isoform2, generated by alternative splicing with an alternate promoter, is a truncated FOXO3 protein with 453 aa that are encoded by exon 2 (FIG. 6B, 6C).

The coding sequences of the mouse and human FOXO3 are highly conserved, determined by 95% of identical amino acids (FIG. 7). When comparing the coding and amino acid sequences of the human FOXO3 isoform2 with the mouse truncated Foxo3 in Foxo3^{flox/flox}; LysMcre* BMMs, we found that 96% of the amino acids are identical (FIG. 8). These new findings indicate that the mouse truncated Foxo3 in Foxo3^{flox/flox}; LysMcre⁺ BMMs is a mouse ortholog of human FOXO3 isoform2. We therefore name this novel Foxo3 isoform as mouse Foxo3 isoform2.

The biological function of the human FOXO3 isoform2 is unclear. Because the Foxo3^{flox/flox}; LysMcre⁺ mice express Foxo3 isoform2 instead of the full-length protein, we hereafter refer to these mice as Foxo3 isoform2 mice, which could be useful as a promising model for studying the function of the newly identified Foxo3 isoform2.

### Mouse Foxo3 Isoform2 Suppresses Osteoclastogenesis and Leads to the Osteopetrotic Phenotype in Mice

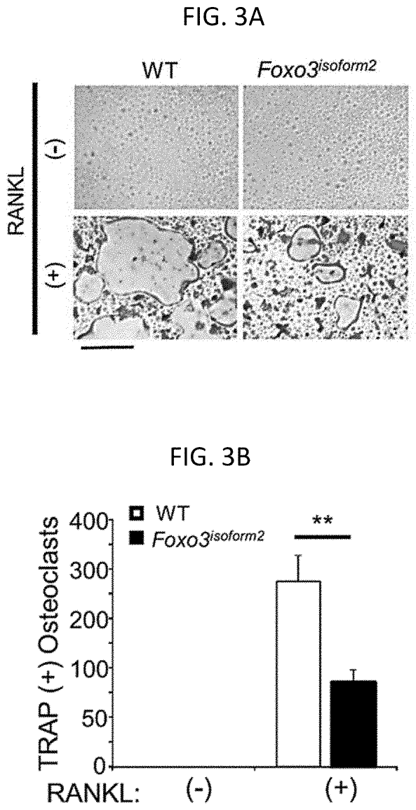

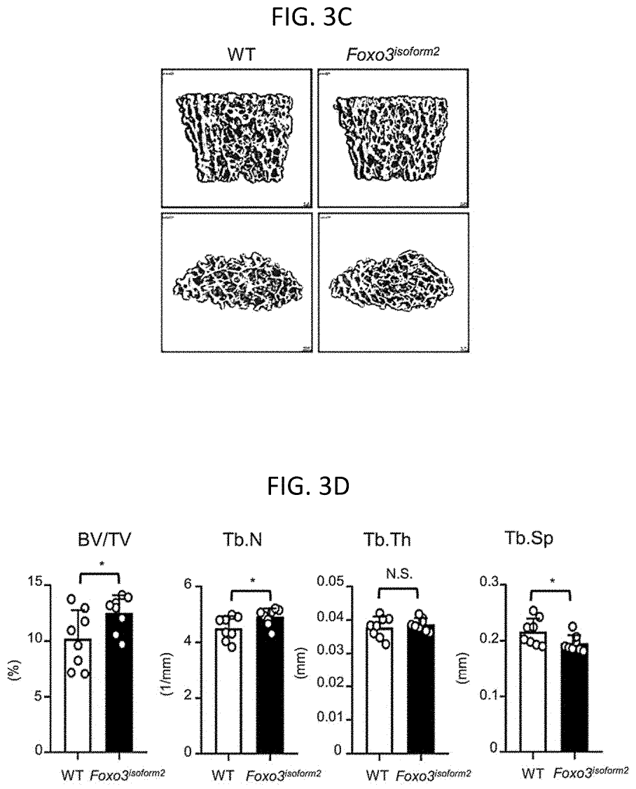

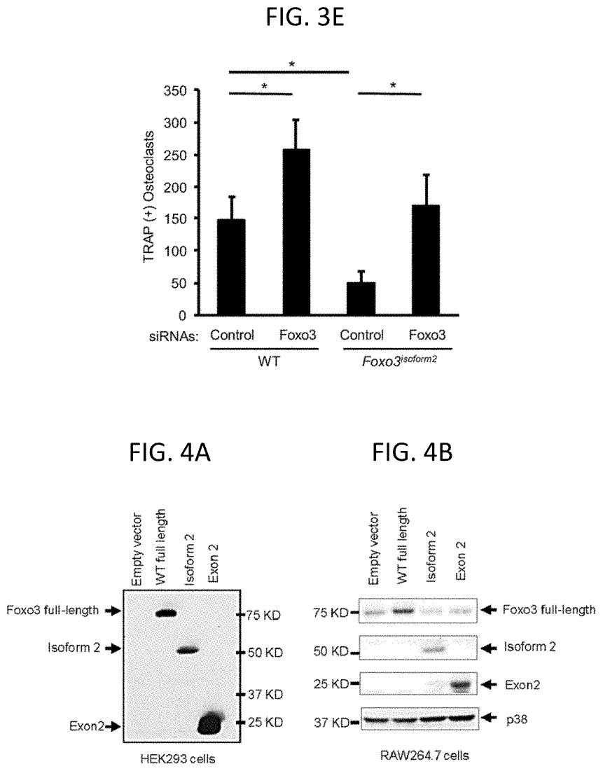

To investigate the role of Foxo3 isoform2 in osteoclastogenesis, we used BMMs as osteoclast precursors to examine osteoclast differentiation in response to RANKL. As shown in FIG. 3A, 3B, the osteoclast differentiation indicated by TRAP-positive multinucleated osteoclast formation induced by RANKL was significantly suppressed in Foxo3 isoform2 BMM cell cultures compared with the WT littermate control cell cultures (FIG. 3A, 3B).

We next performed microcomputed tomographic (mCT) analyses to examine the bone phenotype of Foxo3 isoform2 mice. The Foxo3 isoform2 mice and their littermate controls exhibit similar body weight and body length (data not shown). As shown in FIG. 3C, 3D, Foxo3 isoform2 mice show an osteopetrotic phenotype indicated by significantly increased trabecular bone volume and number but decreased trabecular bone spacing. Taken together with the suppressed osteoclast differentiation in Foxo3 isoform2 cells, these data demonstrate that expression of Foxo3 isoform2 in mice leads to an osteopetrotic bone phenotype.

Consistent with the Foxo3 global KO data (FIG. 1), knockdown of Foxo3 using RNA interference in WT BMMs enhanced osteoclast differentiation (FIG. 3E). Furthermore, knockdown of Foxo3 isoform2 in Foxo3 isoform2 BMMs significantly elevated osteoclastogenesis (FIG. 3E), supporting the inhibitory role of Foxo3 isoform2 in osteoclast differentiation.

We next performed a structure-functional analysis of Foxo3 protein in osteoclast differentiation. We cloned and generated a series of plasmids that express full-length WT Foxo3 or recombinant Foxo3 peptides encoded by the isoform2 or by exon 2 (hereafter referred to as Exon 2). We confirmed the protein expression of each plasmid in HEK293 cells (FIG. 4A) and RAW264.7 cells (FIG. 4B) after transfection. As shown in FIG. 4C, RANKL induced osteoclast differentiation in the RAW264.7 cells transfected with empty vector. Overexpression of WT full-length Foxo3 or isoform2 drastically inhibited osteoclast differentiation. The isoform2 seems to possess a stronger inhibitory effect on osteoclast differentiation than the full-length protein. Interestingly, expression of exon 2 significantly promoted osteoclast differentiation (FIG. 4C). These data were further corroborated by the corresponding changes in osteoclast marker gene expression, such as TRAP and cathepsin K (FIG. 4D). Because isoform2 is encoded by exon 3, these results argue that exon 3 is mainly responsible for osteoclastic inhibition, whereas exon 2 likely counteracts this effect.

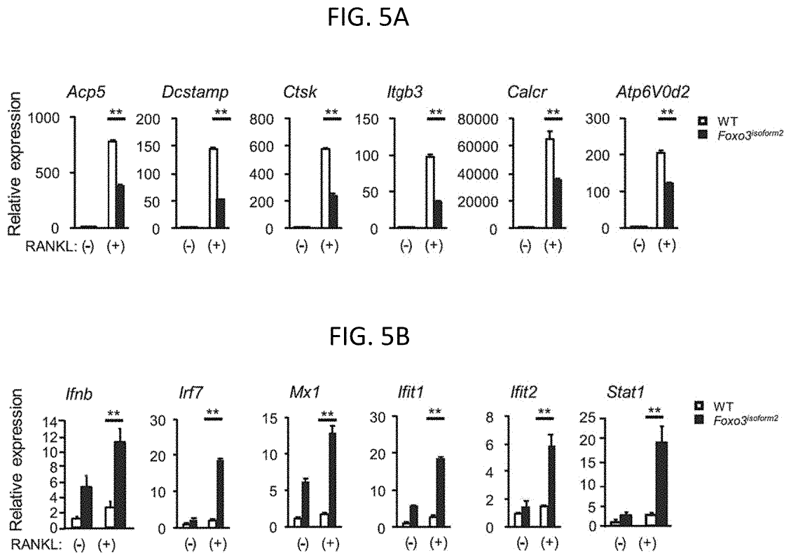

Foxo3 isoform2 represses osteoclast differentiation via endogenous type I IFN-mediated feedback inhibition We next set off to explore the mechanisms by which Foxo3 isoform2 inhibits osteoclastogenesis. In parallel with the suppressed generation of TRAP-positive polykaryons, we found that the expression of osteoclast marker genes Acp5 (encoding TRAP), Ctsk (encoding cathepsin K), Itgb3 (encoding b3 integrin), Dcstamp (encoding Dc-Stamp), Calcr (encoding calcintonin receptor), and Atp6V0d2 (encoding ATPase H+ Transporting V0 Subunit D2) was drastically decreased in RANKL-treated Foxo3 isoform2 cells relative to the WT control cells (FIG. 5A). A previous study shows that Foxo3 targets catalase and Cyclin D1 to arrest cell cycle and promote apoptosis in RANKL-induced osteoclastogenesis (16).

Such Foxo3-mediated changes, however, were not detected in the Foxo3 isoform2 osteoclastogenesis (data not shown). In contrast, we found that the expression of Irf7, an IFN-responsive gene, was markedly elevated in RANKL-treated Foxo3 isoform2 cells relative to WT control cells (FIG. 5B). IRF7 has been identified as a Foxo3 target (29). It is also well established that endogenous IFN-β produced by osteoclast precursors is a strong feedback mechanism that restrains osteoclastogenesis (5, 30, 31). We therefore asked whether the inhibitory effects of Foxo3 isoform2 involves type I IFN-mediated inhibition. Previous studies showed that RANKL treatment can induce a low level of IFN-β expression in macrophages/osteoclast precursors. Although the magnitude of type I IFN induction by RANKL is small (10 pg/ml after 24 h stimulation) when compared with other stimuli such as TLR stimulation, the high potency of type I IFN effects allow these small concentrations to inhibit osteoclast differentiation (30, 31). Consistent with these observations (30, 31), we found that RANKL induced IFN-β expression in WT BMMs and Foxo3 isoform2significantly increased IFN-b induction (FIG. 5B). The enhancement of IFN expression by Foxo3 isoform2 was further corroborated by the elevated expression of IFN-responsive genes, such as M×1, Ifit1, Ifit2, Irf7, and Stat1 after RANKL treatment (FIG. 5B).

These results clearly demonstrate enhanced Ifnb expression and response by Foxo3 isoform2 during osteoclastogenesis and indicate that Foxo3 isoform2 suppresses osteoclastogenesis via type I IFN-mediated feedback inhibition.

### Example 3: Discussion

Similarly to the other Foxo proteins, the function of Foxo3 is largely regulated through posttranslational modifications, such as phosphorylation, acetylation, methylation, and ubiquitination. These posttranslational modifications are context dependent and create a complex set of codes, which affect the subcellular location of Foxo3 and give rise to the diverse functions of Foxo family proteins in response to different stimuli (10-14). For example, Foxo3 can be phosphorylated by various protein kinases at many phosphorylation sites from the N to C terminus of the protein. Phosphorylation of specific sites by kinases, such as AKT, SGK1, CDK2, ERK, and IKK, induces cytoplasmic translocation and/or degradation of Foxo3, leading to target gene inhibition. In contrast, phosphorylation of the activating sites by kinases MST1, JNK, and AMPK usually leads to nuclear localization of Foxo3 and the activation of its target genes (10-14, 32). Foxo3 isoform2 lacks most of the N-terminal DNA-binding domain while maintaining the nuclear localization signal, the C-terminal nuclear export signal, and the transactivation domain at C terminus. This molecular structure implies that Foxo3 isoform2 is likely to lose the direct transcriptional regulation of the genes targeted by the full-length Foxo3 because of the lack of DNA-binding domain. However, Foxo3 isoform2 holds several activating phosphorylation sites that usually contribute to gene activation. In addition to the direct DNA-binding transcriptional activity, Foxo transcription factors are able to regulate transcription in a DNA-binding independent manner, often by interaction with other transcriptional activators or repressors. Hence, we cannot exclude the possibility that Foxo3 isoform2 regulates gene transcription in the nucleus together with other partners. In addition, Foxo3 isoform2 carries the nuclear localization signal as well as the nuclear export signal that allow it to shuttle between the nucleus and cytoplasm in response to environmental cues. The overall impact from these possibilities will determine the subcellular localization of Foxo3 isoform2 and the mechanisms by which it inhibits osteoclastogenesis. The exon 2 peptide is shown to promote osteoclast differentiation. With the consideration that exon 2 contains an N-terminal DNA-binding domain, the direct DNA binding presumably results in the osteoclastogenic activity of exon 2, which in turn attenuates the full-length Foxo3's ability in osteoclast inhibition. Further experiments are needed to elucidate the shared or distinct mechanisms mediated by full-length Foxo3 and the isoform2.

Protein isoforms from the exon skipping mode of alternative splicing often end up with a lack of certain domains that distinguish the function of the isoforms from their original full-length proteins. For example, previous studies identify IRF7 as a critical direct target of FOXO3, and FOXO3 negatively regulates IRF7 transcription in the antiviral response (29). Our results show that Foxo3 isoform2 expression elevates Irf7 transcription and corresponding type I IFN response during osteoclastogenesis. Foxo3 isoform2 lacks the DNA-binding domain and thus may function as an activator to increase Irf7 expression in a DNA-binding independent manner. Although Irf7 is a common target by both full-length Foxo3 and the isoform2, they show distinct regulatory effects on Irf7 expression presumably because of their different DNA binding capacity. Our results revealed that Foxo3^{flox/flox};LysMcre⁺ mice are not fully conditional KO mice because of the existence of the isoform2. The position of the loxp sites caused an in-frame deletion of exon 2 in this mouse line. This was not known at the time when previous loss-of-function studies used this Foxo3^{flox/flox} line. The interpretation of the mutant phenotype in such studies might be now questionable, dependent on cell types. Therefore, future work should pay close attention to the verification of frameshift deletion by cre-loxp recombination as well as the presence of isoforms. Collectively, our findings in the current study identify the first, to our knowledge, known biological function of Foxo3 isoform2, which acts as an important suppressor of osteoclast differentiation via endogenous type I IFN-mediated feedback inhibition. The Foxo3 floxed allele mice (Foxo3^{flox/flox}) could be used as a mouse resource in various areas to investigate the function of Foxo3 isoform2 that recapitulates human FOXO3 isoform2. Environmental cues often affect gene transcription and alternative splicing. For example, bone marrow macrophages/osteoclast precursors mainly express full-length Foxo3 with a trace amount of isoform2 in a physiological condition. Upon RANKL stimulation, Foxo3 isoform2 expression is increased (FIG. 2), which contributes to osteoclastic feedback inhibition. Thus, we speculate that the expression patterns and functions of Foxo3 isoform2 may be altered in response to different environmental settings. It will be of particular interest and clinical relevance to investigate the expression levels and functions of FOXO3 isoform2 in human cells, for instance, in human osteoclasts in healthy conditions versus disease settings, such as in osteoporosis and RA.

### REFERENCES

1.Novack, D. V., and S. L. Teitelbaum. 2008. The osteoclast: friend or foe? Annu. Rev. Pathol. 3: 457-484.
2. Sato, K., and H. Takayanagi. 2006. Osteoclasts, rheumatoid arthritis, and osteoimmunology. Curr. Opin. Rheumatol. 18: 419-426.
3. Schett, G., and E. Gravallese. 2012. Bone erosion in rheumatoid arthritis: mechanisms, diagnosis and treatment. Nat. Rev. Rheumatol. 8: 656-664.
4. Asagiri, M., and H. Takayanagi. 2007. The molecular understanding of osteoclast differentiation. Bone 40: 251-264.
5. Binder, N., C. Miller, M. Yoshida, K. Inoue, S. Nakano, X. Hu, L. B. Ivashkiv, G. Schett, A. Pemis, S. R. Goldring, et al. 2017. Def6 restrains osteoclastogenesis and inflammatory bone resorption. J. Immunol. 198: 3436-3447.
6. Li, S., C. H. Miller, E. Giannopoulou, X. Hu, L. B. Ivashkiv, and B. Zhao. 2014. RBP-J imposes a requirement for ITAM-mediated costimulation of osteoclastogenesis. J. Clin. Invest. 124: 5057-5073.
7. Zhao, B., S. N. Grimes, S. Li, X. Hu, and L. B. Ivashkiv. 2012. TNF-induced osteoclastogenesis and inflammatory bone resorption are inhibited by transcription factor RBP-J. J. Exp. Med. 209: 319-334.
8. Zhao, B., and L. B. Ivashkiv. 2011. Negative regulation of osteoclastogenesis and bone resorption by cytokines and transcriptional repressors. Arthritis Res.

Ther. 13: 234.
9. Zhao, B., M. Takami, A. Yamada, X. Wang, T. Koga, X. Hu, T. Tamura, K. Ozato, Y. Choi, L. B. Ivashkiv, et al. 2009. Interferon regulatory factor-8 regulates bone metabolism by suppressing osteoclastogenesis. Nat. Med. 15: 1066-1071.
10. Hedrick, S. M., R. Hess Michelini, A. L. Doedens, A. W. Goldrath, and E. L. Stone. 2012. FOXO transcription factors throughout T cell biology. Nat. Rev. Immunol. 12: 649-661.
11. Tia, N., A. K. Singh, P. Pandey, C. S. Azad, P. Chaudhary, and I. S. Gambhir. 2018. Role of Forkhead Box 0 (FOXO) transcription factor in aging and diseases. Gene 648: 97-105.
12. Wang, X., S. Hu, and L. Liu. 2017. Phosphorylation and acetylation modifications of FOXO3a: independently or synergistically? Oncol. Lett. 13: 2867-2872.
13. Salih, D. A., and A. Brunet. 2008. FoxO transcription factors in the maintenance of cellular homeostasis during aging. Curr. Opin. Cell Biol. 20: 126-136.
14. van der Vos, K. E., and P. J. Coffer. 2008. FOXO-binding partners: it takes two to tango. Oncogene 27: 2289-2299.
15. Morris, B. J., D. C. Willcox, T. A. Donlon, and B. J. Willcox. 2015. FOXO3: a major gene for human longevity—A mini-review. Gerontology 61: 515-525.
16. Bartell, S. M., H. N. Kim, E. Ambrogini, L. Han, S. Iyer, S. Serra Ucer, P. Rabinovitch, R. L. Jilka, R. S. Weinstein, H. Zhao, et al. 2014. FoxO proteins restrain osteoclastogenesis and bone resorption by attenuating H₂O₂ accumulation. Nat. Commun. 5: 3773.
17. Wang, Y., G. Dong, H. H. Jeon, M. Elazizi, L. B. La, A. Hameedaldeen, E. Xiao, C. Tian, S. Alsadun, Y. Choi, and D. T. Graves. 2015. FOXO1 mediates RANKL-induced osteoclast formation and activity. J. Immunol. 194: 2878-2887.
18. Gregersen, P. K., and N. Manjarrez-Orduño. 2013. FOXO in the hole: leveraging GWAS for outcome and function. Cell 155: 11-12.
19. Lee, J. C., M. Espei, C. A. Anderson, M. A. Linterman, J. M. Pocock, N. J. Williams, R. Roberts, S. Viatte, B. Fu, N. Peshu, et al; UK IBD Genetics Consortium. 2013. Human SNP links differential outcomes in inflammatory and infectious disease to a FOXO3-regulated pathway. Cell 155: 57-69.
20. Miller, C. H., S. M. Smith, M. Elguindy, T. Zhang, J. Z. Xiang, X. Hu, L. B. Ivashkiv, and B. Zhao. 2016. RBP-J-regulated miR-182 promotes TNF-a-induced osteoclastogenesis. J. Immunol. 196: 4977-4986.
21. Vacik, T., and I. Raska. 2017. Alternative intronic promoters in development and disease. Protoplasma 254: 1201-1206.
22. Kim, H. K., M. H. C. Pham, K. S. Ko, B. D. Rhee, and J. Han. 2018. Alternative splicing isoforms in health and disease. Pflugers Arch. 470: 995-1016.
23. Dlamini, Z., F. Mokoena, and R. Hull. 2017. Abnormalities in alternative splicing in diabetes: therapeutic targets. J. Mol. Endocrinol. 59: R93-R107.
24. Inoue, K., Z. Deng, Y. Chen, E. Giannopoulou, R. Xu, S. Gong, M. B. Greenblatt, L. S. Mangala, G. Lopez-Berestein, D. G. Kirsch, et al. 2018. Bone protection by inhibition of microRNA-182. Nat. Commun. 9: 4108.
25. Trapnell, C., L. Pachter, and S. L. Salzberg. 2009. TopHat: discovering splice junctions with RNA-Seq. Bioinformatics 25: 1105-1111.
26. Trapnell, C., B. A. Williams, G. Pertea, A. Mortazavi, G. Kwan, M. J. van Baren, S. L. Salzberg, B. J. Wold, and L. Pachter. 2010. Transcript assembly and quantification by RNA-Seq reveals unannotated transcripts and isoform switching during cell differentiation. Nat. Biotechnol. 28: 511-515.
27. Anders, S., P. T. Pyl, and W. Huber. 2015. HTSeq—a Python framework to work with high-throughput sequencing data. Bioinformatics 31: 166-169.
28. Robinson, M. D., D. J. McCarthy, and G. K. Smyth. 2010. edgeR: a Bioconductor package for differential expression analysis of digital gene expression data. Bioinformatics 26: 139-140.
29. Litvak, V., A. V. Ratushny, A. E. Lampano, F. Schmitz, A. C. Huang, A. Raman, A. G. Rust, A. Bergthaler, J. D. Aitchison, and A. Aderem. 2012. A FOXO3-IRF7 gene regulatory circuit limits inflammatory sequelae of antiviral responses.

Nature 490: 421-425.
30. Takayanagi, H., S. Kim, K. Matsuo, H. Suzuki, T. Suzuki, K. Sato, T. Yokochi, H. Oda, K. Nakamura, N. Ida, et al. 2002. RANKL maintains bone homeostasis through c-Fos-dependent induction of interferon-beta. Nature 416: 744-749.
31. Yarilina, A., K. H. Park-Min, T. Antoniv, X. Hu, and L. B. Ivashkiv. 2008. TNF activates an IRF1-dependent autocrine loop leading to sustained expression of chemokines and STAT1-dependent type I interferon-response genes. Nat. Immunol. 9: 378-387.
32. Calnan, D. R., and A. Brunet. 2008. The FoxO code. Oncogene 27: 2276-2288.

What is claimed is:

1. A method of suppressing osteoclast differentiation or function and/or bone resorption or destruction in a subject in need thereof, comprising increasing the amount, expression, or activity of Foxo3 isoform 2 in the subject.

2. A method of treating a skeletal disease in a subject in need thereof, the method comprising increasing the amount, expression, or activity of Foxo3 isoform 2 in the subject.

3. The method of claim 1 or 2, wherein Foxo3 isoform 2 has the sequence of SEQ ID NO: 1 or a sequence sharing at least 90% identity therewith.

4. The method according to any one of claims 1 to 3, comprising administering an agonist of Foxo3 isoform 2, or a functional fragment thereof.

5. The method according to any one of claims 1 to 3, comprising administering a nucleic acid which comprises a sequence encoding Foxo3 isoform 2 having the sequence of SEQ ID NO: 1 or a sequence sharing at least 90% identity therewith, or a functional fragment of Foxo3 isoform 2, having a N-terminal truncation and sharing at least 90% identity with SEQ ID NO: 1.

6. The method according to any one of claims 1 to 3, comprising administering a polypeptide having the sequence of SEQ ID NO: 1 or a sequence sharing at least 90% identity therewith, or a functional fragment of Foxo3 isoform 2, having a N-terminal truncation and sharing at least 90% identity with SEQ ID NO: 1.

7. A pharmaceutical composition comprising a pharmaceutically acceptable carrier, diluent, or excipient and a viral vector comprising a nucleic acid which comprises a sequence encoding Foxo3 isoform 2 or a sequence sharing at least 90% identity therewith, or a functional fragment of Foxo3 isoform 2, having a N-terminal truncation and sharing at least 90% identity with SEQ ID NO: 1.

8. The composition according to claim 7, wherein the viral vector is an adenoviral vector or AAV vector.

9. The composition according to claim 7 or claim 8, wherein the nucleic acid comprises SEQ ID NO:2, or a sequence sharing at least 70% identity therewith.

10. A pharmaceutical composition comprising a pharmaceutically acceptable carrier, diluent, or excipient and a polypeptide having the sequence of SEQ ID NO: 1 or a sequence sharing at least 90% identity therewith, or a functional fragment of Foxo3 isoform 2, having a N-terminal truncation and sharing at least 90% identity with SEQ ID NO: 1.

11. A method of assessing the efficacy of a treatment comprising measuring the level of Foxo3 isoform 2 in the blood of a subject receiving treatment, wherein an increase in the level of Foxo3 isoform 2 indicates effectiveness of the treatment for treating a skeletal disease.

12. A method of diagnosing an increased risk of developing a skeletal disease in a subject, the method comprising measuring the level of Foxo3 isoform 2 in the blood of a subject receiving treatment, wherein a decrease in the level of Foxo3 isoform 2 as compared to a control level indicates a greater risk of developing a skeletal disease.

13. The method of claim 12, wherein a level of 100 ng/mL or lower is indicative of an increased risk of a skeletal disease in the subject, as compared to a control.

14. A method of diagnosing a skeletal disease in a subject, the method comprising measuring the level of Foxo3 isoform 2 in the blood of a subject receiving treatment, wherein a decrease in the level of Foxo3 isoform 2 as compared to a control level indicates the presence of a skeletal disease.

15. The method of claim 12, wherein a level of 1 ng/mL or lower is indicative of a skeletal disease in the subject, as compared to a control.

16. The method according to any of claims 11-15, wherein the skeletal disease is osteoporosis.
